(19) United States
(12) Patent Application Publication
(10) Pub. No.: US 2019/0249215 A1
(43) Pub. Date: Aug. 15, 2019

(54) Three-Dimensional Cell-Derived Extracellular Matrix/Gel Systems

(71) Applicant: Institute For Cancer Research d/b/a The Research Institute Of Fox Chase Cancer Center, Philadelphia, PA (US)

(72) Inventors: Ruchi Malik, Philadelphia, PA (US); Edna Cukierman, Philadelphia, PA (US); Peter I. Lelkes, Philadelphia, PA (US)

(21) Appl. No.: 16/272,251

(22) Filed: Feb. 11, 2019

Related U.S. Application Data
(60) Provisional application No. 62/629,786, filed on Feb. 13, 2018.

(51) Int. Cl. C12Q 1/02; C12N 5/00; C12N 5/09

(52) U.S. Cl. CPC C12Q 1/025; C12N 5/0018; C12N 5/0693; G01N 2800/56; C12N 2533/90; G01N 2800/52; G01N 2800/7028

(57) ABSTRACT

The present disclosure provides extracellular matrix (ECM)/gel systems that have a substrate having a gel on a surface thereof, and a cell-derived matrix (CDM) on the surface of the gel, and methods of making ECM/gel systems, and methods of using ECM/gel systems to, for example, screen agents for anti-cancer or anti-fibrotic activity.

### REFERENCE TO GOVERNMENT GRANTS

This invention was made with government support under Grant Nos. CA113451 and CA06927 awarded by the National Institutes of Health, and under Grant No. W81XH-15-1-0170 awarded by the United States Department of Defense. The government has certain rights in the invention.

### FIELD

The present disclosure is directed, in part, to cell-derived extracellular matrix/gel systems and methods of making and using the same.

### BACKGROUND

The 5-year survival of patients with pancreatic ductal adenocarcinoma (PDAC) remains at about 7%. PDAC will become the second most lethal cancer, in the U.S., by 2020. Extracellular matrix (ECM) is a complex mixture of structural proteins, which provide not only essential physical scaffolds to maintain tissue structure but also various biomechanical and biochemical signals to modulate cellular function, such as differentiation, migration, proliferation, and survival. Production, assembly, and remodeling of the ECM are tightly regulated processes. In many epithelial cancers, such as in PDAC, loss of homeostatic equilibrium in the normal stroma (i.e., tissue areas that are not cancer) undergo a series of changes due to tumor associated or tumor predisposing chronic inflammation. These inflammatory stresses enable mechanical and biochemical changes that result in the formation of an activated/pathological fibrotic stroma, known as “desmoplasia.” PDAC's desmoplasia typically encompasses about 80% of the resected tumor mass (i.e., surgically resected). Desmoplasia includes ECM remodeling which results in the formation of highly anisotropic matrices, recognized by imaging of collagen fibers. This desmoplastic ECM characteristic has been shown to correlate with poor patient survival, yet the particular tumor-promoting mechanisms that are associated with desmoplasia remain unclear. Along with the alterations observed in the stromal compartment, over 90% of all PDAC cases include a KRAS activating mutation in the tumor cells. Activating KRAS mutations occur early during PDAC development and are essential for cancer initiation and progression. Also, fibrotic stromal predispositions, such as pancreatitis, support tumor development in genetic KRAS-mutated animal model systems. Importantly, there is a known signaling reciprocity between tumor and stroma. For example, oncogenic KRAS transmits signals to activate or promote the activation of stroma, which in turn reciprocate back to exacerbate KRAS-driven tumorigenesis. Recent studies have shown that PDACs bearing activating KRAS mutations (e.g., KRAS^{G12D}) are contingent on exogenous chronic stimulation driven by desmoplasia. Thus, pancreatic desmoplastic ECM play a key role in PDAC progression, however it is unclear which microenvironmental cues of the ECM are needed to inhibit pro-tumorigenic effects of pancreatic desmoplastic stroma.

Relevant to the present disclosure, PDAC and its desmoplasia represent an example for desmoplasia bearing epithelial cancers (like lung, colon, breast, and many others) as well as for non-cancerous fibrosis. Additionally, there is a clear need for better 3D model systems that could accurately mimic the natural stromal environment and incorporate the stromal dynamic changes that are seen in vivo.

It is well known that activated fibroblasts (relevant to cancer CAFs), albeit under tumor signal reciprocity, are responsible for producing the desmoplastic stromal ECM. Recent studies have shown that ablation of CAFs is detrimental to patients. Therefore, reprogramming CAFs, to maintain their tumor suppressive capabilities and re-gain the natural tumor restrictive aspects of stroma, are strongly sought out outputs in designing new therapies. The same is true with fibrosis; there is a clinical need to re-gain the natural function of the organ as opposed to ablating the cells. A critical factor that regulates CAFs' ability to alter and maintain desmoplastic stroma (or activated myofibroblast in fibrosis) is the stiffness of their self-produced and remodeled ECM. Changes in tissue stiffness, mostly observed in the stroma, can impact normal fibroblastic cells as well as activated myofibroblastic (i.e., CAF) contractility and these cells' ability to dynamically remodel the ECM through mechano-sensitive pathways such as the ones including nuclear YAP. Increase in CAF contractility in response to stiffness further promotes changes in ECM architecture, such as its alignment on a forward feedback loop creating a field expansion of desmoplasia or fibrosis (i.e. dynamic reciprocity). Studies have suggested that pancreatic tumor stiffness is approximately 2.5-fold greater than the physiological stiffness of the healthy pancreas. Despite reports suggesting that mechanical properties of stroma, including its ECM, play central roles in regulation of tumorigenesis, the particular biomechanical mechanisms that enable stromal ECM production with tumor restrictive capabilities remain elusive. Hence, the use of cell-derived matrix (CDM)/gel systems described herein could facilitate these types of studies.

Biomechanical manipulations of fibroblastic cells (including CAFs) could alter their behavior in a way that it could activate naïve fibroblasts to become myofibroblastic/CAFs or relax CAFs to behave back like normal. The later could serve to induce “normalization” providing a tumor restrictive benefit as opposed to CAF promoting tumorigenesis. This idea is of noteworthy interest in the field and could be applicable for treating desmoplastic bearing cancers like PDAC as well as chronic cellular fibrosis diseases.

This subject matter disclosed herein takes under consideration the fact that for accurate in vivo mimicry in the laboratory, cells need to be cultured onto surfaces that match the natural stiffness (or softness) of the tissues of origin, as opposed to culturing these cells onto stiff plastic or glass. Hence, many investigators have opted to use different two-dimensional (2D) hydrogels with the goal of matching physiological or pathological stiffness of interest. One limitation of such materials and approaches is that the cells are cultured on top as opposed to within these materials, so these “gels” are mostly 2D. To overcome this limitation, some investigators have used synthetic materials of tunable stiffnesses that serve as three-dimensional (3D) scaffolds in which cells can be cultured. Synthetic scaffolds, however, have the limitation that the scaffolding materials do not consist of the natural fibrous polymer combinations produced by local cells (for each tissue). Not even the use of single natural materials like collagen gels suffices because these too lack the convoluted architectural and biochemical composition of the various natural ECMs. Architectural and material compositions provide signal transduction cues into the cells (i.e., mechanical and biochemical signals via receptors localized on the plasma membrane of the cell). Therefore, selecting the correct materials for the scaffolds is critical for refining questions being asked and accurate interpretation of data obtained using these approaches. To this end, efforts (including our own published data) have been conducted to develop systems that can accurately recapitulate architectural 3D fibrous environment in which fibroblastic cells reside in vivo with the goal of using these to study ECM production mechanisms as well as effects imparted by various ECMs on cells residing within these. To this end fibroblastic cell-derived ECMs, or CDMs, constitute an ideal model because the type of ECM being produced by fibroblastic cells depends on the type of fibroblastic cell being used and it recapitulates many of the in vivo sought out properties. Then again, CDMs have a limitation that they are very thin and because these are produced onto stiff glass or plastic the stiffness of the underlying substrate has an effect on cells when cultured within CDMs.

The following constitutes information and limitations of currently available gels and matrix systems used by the research field. These include inert stiffness-tunable gels, synthetic scaffolds, natural scaffolds/gels, CDMs, and decellularized tissues/organs.

Inert stiffness-tunable gels, such as polyacrylamide gels, offer advantages in controlling stiffness and are relatively easy and cheap to produce in large quantities. This type of system, however, is 2D (cells are cultured on top of these) and necessitates coating using matrix proteins because the materials are inert, and cells will not grow on or adhere onto these. Hence, cells are cultured onto these types of gels in a 2D manner similar to how cells are cultured onto petri dishes with the difference of tuning the underlying stiffness of the culturing flat surface area. Consequently, cells are cultured on top of a flat surface and cannot penetrate the gel. Although synthetic scaffolds offer excellent control over stiffness and fiber/scaffold architecture (i.e., fiber alignment), these scaffolds are generally composed of non-biological material and, thus, do not often mimic in vivo conditions.

Natural scaffolds/gels can be formed using materials that polymerize spontaneously, such as collagen, Matrigel, or Fibrin gels. In some instances, these materials are used as natural-like scaffolds and can account for some of the architecture and stiffness seen in tissues. Nonetheless, albeit these are indeed natural materials, a major disadvantage of these systems is that they lack the in vivo biochemical complexity.

A major advantage of a mesenchymal CDM system is that the ECMs are naturally synthesized and organized by fibroblastic cells. We (and others) have demonstrated in numerous publications that fibroblastic CDMs vary according to the type of fibroblastic cell used (i.e., naive/homeostatic or pathological/activated) and effectively mimic both matrix components and architectural characteristics. Although CDMs offer the sought out biochemical complexity and spatial organization of ECM scaffolds that cells experience in vivo, a disadvantage is that CDMs are very thin (usually between 5 and 15 microns thick). It has been proven that at least 100 microns are needed for cells to evade “sensing” the underlying stiffness of the glass or plate used for matrix production. Therefore, CDMs produced onto glass coverslips (or similar) suffer from a gradient stiffness effect and only the top matrix layers are in vivo like. This fact renders irreproducibility between CDM batches and constitutes a major limitation; this is why canonical CDMs are often referred to as “2.5D” as opposed to “3D” in the literature.

Decellularized tissues and organs can also be used as scaffolds. Although these are physiologically accurate and include the architectural and biochemical complexity of in vivo, these are not practically available and it is difficult to obtain sufficient quantity while these are often denatured during the decellularization process rendering them “altered.”

### SUMMARY

Given the limitations of the above-listed systems, we thought it would be of great interest to develop a hybrid system that combines the advantages of CDMs, providing in vivo like architectural and biochemical complexity, with 2D hydrogels that enable culturing cells isolated from the stiffness of the culturing plate and that can provide an underlying physiological or pathological stiffness in which to produce CDMs. Thus, we developed the combined “CDM/gel system” that allows production of ECMs on top of gels of tunable stiffnesses. We applied this system to examine how to manipulate CAF CDMs and render tumor-suppressive CDM materials and to identify specific biomechanical/mechano transduction mechanisms associated with CDM-imparted tumor restrictions using in PDAC cells as a proof of principle model. As described herein, CDMs obtained from patient harvested pancreatic CAFs that are produced onto 2D polyacrylamide gels of stiffnesses the physiological (about 1.5 kPa) and pathological (about 7.5 kPa) pancreas have been successfully generated and characterized.

The present disclosure provides extracellular matrix (ECM)/gel systems comprising: a substrate having a gel on a surface thereof; and a cell-derived extracellular matrix (CDM) on the surface of the gel. In some embodiments, the gel is a polyacrylamide gel. In some embodiments, the Young's moduli of the gel is from about 0.5 kPa to about 10 kPa. In some embodiments, the cells of the cell-derived extracellular matrix are normal cells or cancer associated cells. In some embodiments, the cancer associated cells are cancer associated fibroblasts.

The present disclosure also provides methods of preparing the ECM/gel systems comprising: forming the gel on a surface of a substrate; and forming the CDM on the surface of the gel. In some embodiments, the gel is formed on the surface of the substrate by a method comprising: chemically activating the substrate; mixing polymer solutions in ratios to provide the gel with a final Young's moduli of from about 0.5 kPa to about 10 kPa to form a polymer solution; initiating polymerization of the polymer solution; and contacting the activated surface of the substrate with the polymer solution. In some embodiments, the CDM is formed on the surface of the gel by a method comprising: adding cell growth media and seeding cells on the surface of the gel; and incubating the substrate having the gel and cells under conditions sufficient for the development of the ECM. In some embodiments, the method further comprises further comprises extracting the CDMs that were produced onto the gels by “denuding” the CDMs from the original matrix producing cells, rendering inert underlying gels containing de-cellularized 3D CDMs attached on top.

The present disclosure also provides methods of screening an agent or a combination of agents for anti-cancer activity or anti-fibrotic activity comprising: contacting the extracellular matrix/gel system with the agent or combination of agents; and assaying the extracellular matrix for at least one anti-cancer characteristic or at least one anti-fibrotic characteristic, whereby if the agent induces at least one anti-cancer characteristic or anti-fibrotic characteristic on the extracellular matrix, the agent is a potential anti-cancer or anti-fibrotic drug. In some embodiments, the agent is a small organic molecule, an antibody, a peptide, or a nucleic acid molecule, or any combination thereof. In some embodiments, the at least one anti-cancer characteristic or anti-fibrotic characteristic of the extracellular matrix is selected from decreased formation of highly anisotropic collagen fibers, decreased stiffness, decrease in cell spheroid migration, decrease in cell proliferation, decreased spindle shaped morphology, decreased levels of alpha-smooth muscle actin, decreased cell-aspect ratio, and decreased matric indentation modulus.

### BRIEF DESCRIPTION OF THE DRAWINGS

FIG. 1 shows epi-fluorescent microscopy images depicting cell shape (Calcein AM; green) and nuclei (blue) of CAFs cultured onto physio (about 1.5 kPa), patho (about 7 kPa) PA gels and coverslip.

FIG. 2 (Panels A, B, C, and D) shows reconstituted maximum projections of confocal images obtained from CDM produced by CAFs cultured on physio and patho substrates, with the graph showing percentages of fibers distributed at 15° angles from the mode for the indicated experimental conditions (Panel A); indentation moduli of decellularized CDMs produced by CAFs cultured onto the indicated substrates (Panel B); mathematical model to validate biphasic distribution in CAF shape as a function of stiffness (Panel C); and mathematical model to validate that anisotropy levels of CAF-derived CDMs (onto stiffness changing gels) are dictated by the biphasic cell's aspect ratios (Panel D).

FIG. 3 (Panels A, B, and C) shows results of Ki67 proliferation assay depicting levels of proliferating KRAS cells on CAF-CDMs generated onto physio or patho stiffness substrates (Panel A); epifluorescence microscopy images showing spread of RFP expressing KRAS spheroids within CAF-CDMs generated on physio versus patho stiffness substrates (Panel B); and indirect immunofluorescence using anti-pERK1/2 antibody (shown in green) and cell nuclei labeled using SYBR green (shown in blue) (Panel C) of the same.

FIG. 4 (Panels A and B) shows immunoblots of nuclear fractions of KRAS cells treated overnight with 20 μM of U0126 to inhibit MEK1/2 upstream to pERK1/2, and phospho-p90RSK, downstream to pERK2, along with an associated graph of output numbers (Panel A); and immunoblots of lysates to assess PDAC (KRAS) area spreads, along with an associated graph of the measured results (Panel B). PDAC/KRAS cells were cultured onto CAF-CDMs generated onto patho gels.

FIG. 5 (Panels A and B) shows merged images of a representative matching normal (physiological) and PDAC (pathological) tissue samples showing epithelium/tumoral cells (red), nuclei (blue), stromal cells (grey) and pERK1/2 (green) for representative examples of pancreatic normal and PDAC samples in vivo (in patients), and associated graph summarizing the generated data outputs of the measured intensity levels (Panel A); and a summary depiction showing that isotropic CDMs (physiologic) direct KRAS mutated and, thus, constitutively activated pERK1/2 (green) to cytosolic locations restricting PDAC cell behaviors (Panel B) as hypothesized and as mimicked using CAF-CDMs produced on physio gels.

FIG. 6 (Panels A, B, C, and D) shows epi-fluorescent microscopy images depicting cell shape (Calcein AM; green) and nuclei (blue) of CAFs cultured onto physio (about 1.5 kPa) or patho (about 7 kPa) PA gels and coverslip, and an associated graph presenting CAF aspect ratios (length/breadth) (Panel A); reconstituted maximum projections of confocal images obtained from CDM produced by control normal fibroblasts cultured on physio and patho substrates, and an associated graph showing percentages of fibers distributed at 15° angles from the mode for the indicated experimental conditions (Panel B); mathematical model to validate that anisotropy levels of CAF-CDMs are dictated by the biphasic cell's aspect ratios (Panel C); and indentation moduli of decellularized CDMs produced by control or CAF fibroblastic cells cultured onto the indicated substrates (Panel D).

FIG. 7 (Panels A and B) shows reconstituted confocal microscopy images obtained from CDMs produced by two independent CAF cells isolated from two different patients; Panel A: cells used through the rest of the study to generate CDMs; and Panel B: additional PDAC patient-derived CAFs (CDMs); CAFs were plated onto the assorted substrates: 3-5 days on gels and 7-8 on glass coverslip; staining of fibronectin (green) and nuclei (blue) and individual monochromatic images are shown (top row); inserts represent only the bottom layers; middle row includes the same CDM images in monochromatic depictions, omitting the nuclei channel; colors in bottom panels depict angle distributions of CDMs, obtained using ‘OrientationJ’ plugin of Image-J software in which HUE was used to normalize colors to include, cyan, as the mode fibers for fiber angle distribution visualization, as indicated by the gradient color bar on the right; graphs indicate percentage of CDM fibers oriented at 15 degrees from the mode measured angle; asterisks denote: * p<0.05 and **** p<0.001.

FIG. 8 (Panels A and B) shows plots of Ki67 levels assessed via indirect immunofluorescence using nuclei stain counts for normalization purposes regarding syngeneic human pancreatic epithelial (hTERT) and cancer (KRAS) cells cultured within the assorted 3D extracted CDMs or onto 2D substrate controls for 24 hours (Panel A); and experimental images of the spreading of RFP expressing KRAS spheroids, of known even sizes, on assorted CDMs for 48 hours, and an associated graph showing relative area spread (Panel B).

FIG. 9 (Panels A, B, and C) shows a Western blot depicting pERK1/2 and total ERK1/2 levels in KRAS cells cultured on soft/physio (about 1.5 kPa) or stiff/patho (about 7 kPa) bare gels and onto glass coverslip after overnight incubation (Panel A); indirect immunofluorescence microscopy images of KRAS cells cultured on soft, stiff and coverslip 2D substrates following overnight incubation, along with an associated graph (Panel B); and indirect immunofluorescence microscopy images of KRAS cells cultured overnight within assorted CDMs and stained with pERK1/2 antibody (green) and their nuclei counterstained using SYBR green (blue), along with an associated graph (Panel C).

### DESCRIPTION OF EMBODIMENTS

The terminology used herein is for the purpose of describing particular embodiments only and is not intended to be limiting.

The CDM/gel systems described herein combine the advantages of the inert stiffness-tunable gel systems and the advantages of the CDM systems. Such a combination overcomes the main limitations of both systems and produces a new system that differs from all available currently available systems. The new combined system overcomes the 2D vs. 3D limitations as well as the underlying stiffness and also overcomes the material selection for the 3D scaffolding production.

The new methods and systems which grow CDMs are more “3D-like” because it separates the CDMs from the coverslip or glass by much more than the needed 100 microns. In addition, the underlying gel (acrylamide) is tunable, thus providing cells with physiological or pathological underlying stiffness on which to conduct CDM fibrillogenesis. The resultant system is reproducible, thicker than regular CDMs, homogenous, and pathophysiologically relevant. The generation of the new system was not trivial as the development and technical troubleshooting were laborious.

As demonstrated herein, the new systems provided advantages as it allowed the manipulation of mechanical/architectural characteristics of the ECM and, therefore, can be used for studying mechanotransduction pathways associated with pancreatic cancer (as well as other desmoplasia bearing cancers and chronic cellular fibrosis diseases). For example, culturing CAFs on pancreatic physiological tissue stiffness resulted in significant reduction in CAF-derived ECM alignment, as well as ECM indentation moduli, which are key properties of desmoplastic tissues. In addition, using previously established isogenic benign (hTERT immortalized) and invasive K-Ras^{G12D}-driven pancreatic cancer cells, it has also been demonstrated herein that biomechanical manipulation of CAF-CDMs reversed some of its tumor promoting features, such as promotion of cell growth, invasion, and subcellular localizations of active-ERK1/2. These results also suggest that CAF-CDM-induced PDAC cell responses, such as growth and spheroid dispersion/invasion, were restricted upon inhibition or downregulation of ERK2, but not ERK1. Thus, the systems presented herein allow manipulation of CAF-CDMs in a 3D environment to study mechanisms associated with matrix-informed tumor progression promoting properties of pancreatic desmoplastic stroma. In addition, the studies described herein reveal that manipulation of the underlying substrate, reverts tumorigenic PDAC cell behaviors in an ERK2-dependent manner.

In addition, the new CDM/gel systems described herein are able to overcome the limitation of previously described CDMs. For example, the new CDM/gel systems provide matrices that have more homogeneous architecture (alignment or lack thereof depending on the conditions and cells used) of ECM fibers in the 3D matrix. It has been observed that the heterogeneity in matrix fibers as well as nuclear orientation is reduced in the new CDM/gel systems as opposed to using coverslips. In contrast, analysis of fibers, nuclei, and selected markers reveals the previous heterogeneity of CDMs obtained in the absence of any gel.

Also, the new CDM/gel systems described herein require fewer numbers of days for matrix production (e.g., 3-5 days for gels versus 7-9 days for coverslip) and the resultant CDMs are much thicker. Accordingly, the new CDM/gel systems may be more cost-effective. The new CDM/gel systems described herein may also be adapted for large scale production. Additionally, this system may also be applied to different types of cancers and is not limited to pancreatic cancer. By using different fibroblastic cells (normal or pathologically altered), CDM production could be affected to render tumor permissive or tumor restrictive CDMs. This has additional implications because it may be used as a better drug screening medium for cancer as well as other fibrosis-related diseases.

The present disclosure provides CDM/gel systems comprising: a substrate having a gel on a surface thereof; and a CDM generated on the surface of the gel.

The substrate can be any material or container upon which a gel can be formed. In some embodiments, the substrate is a petri dish, a multi-well plate, or a coverslip. In some embodiments, the multi-well plate is a 6-well plate, an 8-well plate, or a 12-well plate. In some embodiments, the coverslip is a glass coverslip, such as one with an 18 mm diameter. In some embodiments, a coverslip can be placed within a multi-well plate, such as a 12-well multi-well plate. In some embodiments, the substrate is activated. In some embodiments, the substrate is activated with 3-aminopropyltriethoxysilane (APTES) or 3-aminopropyldimethylethoxysilane (APDMES). In some embodiments, the substrate is activated with 3-aminopropyltriethoxysilane (APTES).

In some embodiments, the gel is a polyacrylamide gel, a polydimethylsiloxane (PDMS) gel, or polyethylene glycol (PEG) gel. In some embodiments, the gel is a polyacrylamide gel. In some embodiments, the polyacrylamide gel comprises acrylamide and N,N′-methylenebiacrylamide.

In some embodiments, the gel is at least 100 μm thick. In some embodiments, the gel has a maximum thickness of 1 to 2 mm. In some embodiments, the gel has a thickness of about 100 μm to about 2 mm. In some embodiments, the gel has a thickness of about 100 μm to about 1 mm. In some embodiments, the gel has a thickness of about 100 μm to about 500 μm. In some embodiments, the gel has a thickness of about 100 μm to about 250 μm.

In some embodiments, the Young's moduli of the gel is from about 0.5 kPa to about kPa. In some embodiments, the Young's moduli of the gel is from about 0.5 kPa to about 3 kPa (i.e., that of a physiological or “physio” tissue). In some embodiments, the Young's moduli of the gel is from about 1 kPa to about 2 kPa. In some embodiments, the Young's moduli of the gel is from about 0.9 kPa to about 1.7 kPa. In some embodiments, the Young's moduli of the gel is about 1.5 kPa. In some embodiments, the Young's moduli of the gel is from about 5 kPa to about 10 kPa (i.e., that of a pathological or “patho” tissue). In some embodiments, the Young's moduli of the gel is from about 6 kPa to about 10 kPa. In some embodiments, the Young's moduli of the gel is from about 5.8 kPa to about 10 kPa. In some embodiments, the Young's moduli of the gel is from about 7 kPa to about 8 kPa. In some embodiments, the Young's moduli of the gel is about 7.5 kPa. The desired Young's moduli of the gel can be obtained by varying the ratio of the polymer components of the gel as known by those skilled in the art. For example, preparation of 10 mL of gel solution for 1.5 kPa comprises: 0.75 mL of acrylamide, 0.75 mL of N,N′-methylenebiacrylamide, and 8.5 mL of distilled water. In some embodiments, preparation of 10 mL of gel solution for 7.5 kPa comprises: 2.5 mL of acrylamide, 0.75 mL of N,N′-methylenebiacrylamide, and 6.75 mL of distilled water. Cross-linking agents (TEMED and APS) are added prior to polymerization.

In some embodiments, the gel is conjugated to a protein. In some embodiments, the protein is collagen, laminin, or fibronectin. In some embodiments, the protein is collagen. In some embodiments, the protein is Collagen-I or Collagen-IV. In some embodiments, the protein is Collagen-I. In some embodiments, the protein is Collagen-I is Rat tail Collagen-I. In some embodiments, the gel is conjugated to two or more proteins.

In some embodiments, the system further comprises cell media. In some embodiments, the cell media is cell growth media. In some embodiments, cell media is supplemented with ascorbic acid. In some embodiments, the cell media is fibroblast media. In some embodiments, the fibroblast media comprises high glucose DMEM with 15% Fetal bovine serum, 1% penicillin/streptavidin, 2 mM L-glutamine, supplemented with 50 μg/mL ascorbic acid.

In some embodiments, the cells of the CDM are normal cells or cancer associated cells. In some embodiments, the normal cells are fibroblast cells. In some embodiments, the normal cells are NIH3T3 cells or patient-derived fibroblasts from normal pancreas. In some embodiments, the cancer associated cells are cancer associated fibroblasts. In some embodiments, the cancer associated cells are cancer associated fibroblasts harvested from patient pancreatic cancer, or other cancers such as, for example, kidney, lung, and breast). In some embodiments, the cancer cells that are used for replating within the decellularized CDM/gel systems are double mutated constitutively active KRAS and P53 loss pancreatic cancer cells or benign (HTERT immortalized) pancreatic epithelial cells.

The present disclosure also provides methods of preparing the CDM/gel systems described herein comprising: forming the gel on a surface of a substrate; and forming the CDM on the surface of the gel.

In some embodiments, the gel is formed on the surface of the substrate by a method comprising: chemically activating the substrate; mixing polymers in ratios to provide the gel with a Young's moduli of from about 0.5 kPa to about 10 kPa or from about 1.5 kPa to about 7.5 kPa, to form a polymer solution; initiating polymerization of the polymer solution; and contacting the activated surface of the substrate with the polymer solution.

In some embodiments, the substrate is any of the substrates described herein such as, for example, a petri dish, a multi-well plate, or a coverslip. In some embodiments, the substrate is activated by 3-aminopropyl triethoxysilane (APTES).

In some embodiments, the gel is any of the gels described herein such as, for example, a polyacrylamide gel, a polydimethylsiloxane (PDMS) gel, or polyethylene glycol (PEG) gel. In some embodiments, the polymers are mixed in ratios that provide the gel with a Young's moduli as described above. For example, in some embodiments, the polymers are mixed in ratios that provide the gel with a Young's moduli of from about 0.5 kPa to about 3 kPa. In some embodiments, the polymers are mixed in ratios that provide the gel with a Young's moduli of from about 1 kPa to about 2 kPa. In some embodiments, the polymers are mixed in ratios that provide the gel with a Young's moduli of about 1.5 kPa. In some embodiments, the polymers are mixed in ratios that provide the gel with a Young's moduli of from about 6 kPa to about 9 kPa. In some embodiments, the polymers are mixed in ratios that provide the gel with a Young's moduli of from about 7 kPa to about 8 kPa. In some embodiments, the polymers are mixed in ratios that provide the gel with a Young's moduli of about 7.5 kPa.

In some embodiments, the method further comprises placing a second substrate on top of the polymer solution on the activated surface of the substrate. In some embodiments, the second substrate is a coverslip. In some embodiments, the coverslip is a treated coverslip. In some embodiments, the coverslip is treated with dichlorodimethylsilane (DCDMS). The second substrate serves to make sure the formed gel has a smooth surface for culturing cells.

In some embodiments, gel polymerization is allowed to proceed at room temperature for about 10 to about 15 minutes. In some embodiments, the method further comprises removing the second substrate after polymerization of the gel. In some embodiments, after polymerization, top coverslip is removed, and the gels are placed in PBS buffer within a multi-well plate.

In some embodiments, the method further comprises conjugating the gel with a protein. In some embodiments, the protein is collagen. In some embodiments, the protein is collagen-I. In some embodiments, the protein is cross-linked to the gel. In some embodiments, protein is cross-linked to the gel using sulfosuccinimidyl 6-(4′-azido-2′-nitrophenylamino)hexanoate) (Sulfo-SANPAH). The protein conjugated gels can be kept at 4° C. in PBS buffer up to 2 weeks until ready for further use.

In some embodiments, the CDM is formed on the surface of the gel by a method comprising: adding cell growth media and cells to the gel; and incubating the substrate having the gel and cells under conditions sufficient for the development of the ECM. In some embodiments, the cell growth media is supplemented with ascorbic acid. In some embodiments, the cell growth media is fibroblast media, such as described herein.

In some embodiments, the method further comprises extracting the CDM from the gel. In some embodiments, the CDM is extracted from the gel using an alkaline detergent such as, for example, Triton X-100 (5%) and NH₄OH (20 mM). The extracted matrices can be used for replating cancer cells and also for fibroblasts. In some embodiments, the extraction of the CDMs that were produced onto the gels occurs by “denuding” the CDMs from the original matrix producing cells, rendering inert underlying gels containing de-cellularized 3D CDMs attached on top.

The present disclosure also provides methods of screening an agent or a combination of agents for anti-cancer activity or anti-fibrotic activity comprising: contacting any of the CDM/gel systems described herein with the agent or combination of agents; and assaying the ECM for at least one anti-cancer characteristic or at least one anti-fibrotic characteristic, whereby if the agent induces at least one anti-cancer characteristic or anti-fibrotic characteristic on the ECM, the agent is a potential anti-cancer or anti-fibrotic drug. In some embodiments, the agent to be screened can be added to the culture media of cells plated within the de-cellularized CDM/gel system. Cells can be subjected to wide range of assays to study their behavior.

In some embodiments, the agent is a small organic molecule, an antibody, a peptide, or a nucleic acid molecule, or any combination thereof. In some embodiments, the antibody is a fragment such as, for example, Fab, Fab′, (Fab′)₂, Fv, scFv, diabody, triabody, tetrabody, Bis-scFv, minibody, Fab₂, or Fab₃.

In some embodiments, the at least one anti-cancer characteristic or anti-fibrotic characteristic of the extracellular matrix is selected from decreased formation of highly anisotropic collagen fibers, decreased stiffness, decrease in cell spheroid migration, decrease in cell proliferation, decreased spindle shaped morphology, decreased levels of alpha-smooth muscle actin, decreased cell-aspect ratio, and decreased matric indentation modulus. In some embodiments, the at least one anti-cancer characteristic or anti-fibrotic characteristic of the extracellular matrix is a decrease in cell spheroid migration or a decrease in cell proliferation. Any agent that decreases the presence of any of these characteristics or decreases the rate of formation or occurrence of any of these characteristics is an agent that may be suitable for anti-cancer and/or anti-fibrotic therapy.

The CDM/gel systems described herein can also be used as a general research tool to investigate the role of the ECM in cancer cell or tumor progression. Example 2 herein is such a use.

The present disclosure also provides the use extracellular matrix/gel systems described herein in the screening of an agent or a combination of agents for anti-cancer activity or anti-fibrotic activity. The methods can be carried out by contacting any of the extracellular matrix/gel systems described herein with the agent or combination of agents, and assaying the extracellular matrix for at least one anti-cancer characteristic or at least one anti-fibrotic characteristic, whereby if the agent induces at least one anti-cancer characteristic or anti-fibrotic characteristic on the extracellular matrix, the agent is a potential anti-cancer or anti-fibrotic drug.

In order that the subject matter disclosed herein may be more efficiently understood, examples are provided below. It should be understood that these examples are for illustrative purposes only and are not to be construed as limiting the claimed subject matter in any manner. Throughout these examples, molecular cloning reactions, and other standard recombinant DNA techniques, were carried out according to methods described in Maniatis et al., Molecular Cloning—A Laboratory Manual, 2nd ed., Cold Spring Harbor Press (1989), using commercially available reagents, except where otherwise noted.

### EXAMPLES

### Example 1: Material and Methods

### Cell Lines and Reagents

Human pancreatic CAFs were isolated, characterized, immortalized and authenticated as previously described (Franco-Barraza et al., Current protocols in cell biology, 2016, 71, 10 19 11-10 19 34; and Franco-Barraza et al., eLife, 2017, 6). As control or normal fibroblasts (NFs), we used NIH-3T3s which were obtained from ATCC. All cells were maintained in a humidified incubator at 37° C. and 5% CO₂. All fibroblasts were cultured in Dulbecco's Modified Eagle's Medium (DMEM) supplemented with 10% FBS, 100 U/mL Penicillin, 100 mg/mL Streptomycin and 2 mM L-Glutamine. Isogenic pancreatic ductal epithelial cells, hTERT and KRAS, were from ATCC and cultured in “pancreatic epithelial cell growth medium” consisting of four parts of low glucose DMEM and one part M3 media supplemented with 5% FBS. DMEM was from Mediatech (Manassas, Va.) and FBS from Atlanta Biologicals (Lawrenceville, Ga.).

The MEK inhibitor U0126 (1,4-diamino-2,3-dicyano-1,4-bis[2-aminophenylthio] butadiene) was obtained from Calbiochem (Billerica, Mass.). N,N′-Methylenebisacrylamide (Cat no. M1533), acrylamide (Cat No. A4058), N,N,N′,N′-Tetramethylethylenediamine accelerator (TEMED), ammonium persulfate (APS), 3-Aminopropyl triethoxysilane (APTES), glutaraldehyde and dichlorodimethylsilane (DCDMS) were obtained from Sigma-Aldrich (St. Louis, Mo.). Rat Tail Collagen-I (Cat No. A1048301) and sulfosuccinimidyl 6-(4′-azido-2′-nitrophenylamino)hexanoate) (Sulfo-SANPAH) were obtained from Thermo Fisher Scientific (Waltham, Mass.).

### Preparation of Polyacrylamide Gels

Polyacrylamide (PA) gels were generated using previously described protocols (Tse et al., Current protocols in cell biology, 2010, Chapter 10, Unit 10 16). In brief, circular glass coverslips, 18 mm in diameter were activated using APTES for 10 minutes and washed extensively with distilled water followed by treatment with 0.5% glutaraldehyde for 1 hour. To prepare the gel solutions, acrylamide and N,N′-Methylenebisacrylamide solution were mixed together in distilled water in desired ratios to generate gel precursor solution for predicted Young's moduli of about 1.5 kPa and about 7.5 kPa. Gel polymerization was initiated by addition of crosslinkers −10% w/v ammonium persulfate (ratio 1:1000) and N,N,N′,N′-Tetramethylethylenediamine (ratio 1:10,000). After gentle mixing, 120 μL of the gel solution was pipetted onto the activated coverslips and a DCDMS-treated coverslip was carefully placed on top of the gel solution. Gels were left to polymerize at room temperature for about 10-15 minutes. The top coverslip was then gently lifted, and the gels washed with Milli-Q water and sterilized under a UV lamp for 15 minutes. Covalent conjugation of PA gels with 50 μg/ml collagen-I was performed in 50 mM HEPES buffer, 8.5 pH. Collagen-I was crosslinked to PA gels using Sulfo-SANPAH for 15 minutes under a UV lamp at 365 nm wavelength. Collagen-coated gels were washed extensively with PBS and stored in PBS at 4° C. for up to two weeks. The Collagen-conjugated gels were equilibrated for 30 minutes with fibroblast media at 37° C. prior to cell seeding and initiating CDM production.

In greater detail, the PA gels can be generated as follows. PA gels can be produced by mixing various acrylamide and bis-acrylamide concentrations and inducing free radical polymerization. PA gel modulus of elasticity can be quantified using atomic force microscopy (AFM). Materials: 0.1 M NaOH; distilled H₂O; 3-aminopropyltriethoxysilane (APES); 0.5% (v/v) glutaraldehyde in phosphate-buffered saline; dichlorodimethylsilane (DCDMS); 40% (w/v) acrylamide stock solution; 2% (w/v) bis-acrylamide stock solution; phosphate-buffered saline (PBS); tetramethylethylenediamine (TEMED); and 10% (w/v) ammonium persulfate (APS).

Amino-silanated coverslips can be prepared by placing them on a hot plate and adding 500 μl of 0.1 M NaOH to the coverslip so that the solution covers the entire glass surface. The coverslip can be heated with solution at about 80° C. until the liquid evaporated. The solution should not boil, and there should be a thin semi-transparent film of NaOH remaining on the coverslip after evaporation. Step 1 can be repeated by diluting the NaOH by adding 500 μl of distilled H₂O to the coverslip and heating the solution at 80° C. until the film of NaOH was uniform. The coverslips can be placed in a nitrogen environment to which 200-250 μl of APES was added to the surface of the coverslips. Five minutes can be allowed for the APES to react. The coverslips can be rinsed with distilled H₂O under the distilled H₂O tap to ensure both the top and bottom of the coverslips are rinsed. The coverslips can be placed in distilled H₂O into a petri dish and rinsed twice, each time in about 10 ml of distilled H₂O for 5 minutes each. The second distilled H₂O wash solution can be aspirated and about 10 ml of 0.5% glutaraldehyde in PBS can be added. The solution can be allowed to stand for 30 minutes. The solution can be aspirated, and the coverslips dried with a Kimwipe, by allowing the coverslips to dry naturally in air, or by blowing nitrogen on them.

To prepare chloro-silanated glass slides, using separate glass slides, spread about 100 μl of DCDMS onto each slide in the fume hood. Ensure that the solution coats the entire surface of the slides. Allow to react for up to 5 minutes before removing the excess DCDMS with a Kimwipe and rinse 1 minute under distilled H₂O.

To prepare statically compliant hydrogels: Mix acrylamide and bis-acrylamide to their desired concentrations in either distilled H₂O or PBS. Add 1/100 total volume of APS and 1/1000 total volume of TEMED to gel solutions. Vortex the polymerizing solution. Quickly pipet 25 μl of the gel solution onto the treated side of the chloro-silanated glass slides and add the amino-silanated coverslips with the treated side down. Allow the gel to polymerize for 5 to 30 minutes and monitor the unused solution to determine when the solution is fully polymerized. Remove the bottom glass slide and discard. Place the top coverslip-gel composite in a 35-mm petri dish or 6-well plate in PBS or dH₂O depending on what was used to dilute the acrylamide. Make sure that the gel-coated side faces up. To remove unpolymerized acrylamide rinse twice, each time for 5 minutes in PBS or distilled H₂O depending on what was used to dilute the acrylamide. To store the hydrogels, immerse the hydrogels in water or PBS to keep them hydrated and store them at 4° C.

To prepare matrix protein substrates of varying stiffness for cell culture, the following procedure can be carried out. Materials: 0.2 mg/ml sulfosuccinimidyl-6-(4′-azido-2′-nitrophenylamino)-hexanoate (sulfo-SANPAH); 50 mM HEPES buffer, pH 8.5, filter sterilized; ECM proteins of choice; and distilled H₂O or PBS. Remove dH₂O or PBS from the petri dish with coverslip-gel composite. Add about 500 μl of sulfo-SANPAH solution to the gel surface or enough to cover the entire gel. Place the gel in the 365-nm UV light source at a distance of about 3 inches and expose for 10 minutes. Repeat as necessary should insufficient protein bind, or if the coating does not appear uniform using the methods detailed below. Rinse with 2 ml of 50 mM HEPES at least two to three times to eliminate excess sulfo-SANPAH. Add an appropriate amount of ECM protein to 50 mM HEPES and incubate this solution with the gel overnight at 37° C. Rinse with 2 ml of distilled H₂O or PBS, depending on what was used to dilute the acrylamide. To verify binding amounts, use either fluorescently labeled or radioactively labeled protein to relate the measured signal to the amount of protein from reference standards. Alternatively, antibody-coated bead binding or enzyme-linked immunosorbent assays (ELISA) may be used to confirm protein binding. After confirmation of protein binding, or in parallel cultures, add 1 ml of sterile distilled H₂O or PBS to the petri dish or well and place in the tissue culture hood for 30 minutes under UV for sterilization. Plate cells using standard tissue culture techniques. For isolated cells with only cell-ECM contacts, plate cells at <10⁴ cells/cm² in their standard medium.

### Preparation of Cell-Derived ECM on PA Gels

To produce cell-derived ECMs on the PA gels, we followed our published procedures (Franco-Barraza et al., Current protocols in cell biology, 2016, 71, 10 19 11-10 19 34; and Franco-Barraza et al., eLife, 2017, 6) with some modifications. Pyrex® cloning cylinders (Sigma Aldrich, St. Louis, Mo.), of dimensions 8 mm (height)×8 mm (diameter), were carefully placed at the center of the Collagen I conjugated PA gels and 100 μL of fibroblast growth media containing 4×10⁴ CAFs (or NFs) was placed inside the cloning cylinders. Cylinders were removed after about 1 hour after the cells had attached and the gels were immersed in 1 mL of fibroblast culture medium. The media was supplemented with 50 μg/ml ascorbic acid to obtain unextracted 3D cell-derived ECMs. The same procedure without cloning cylinders was employed for obtaining CDMs directly on the coverslips (Tse et al., Current protocols in cell biology, 2010, Chapter 10, Unit 10 16). The coverslip control CDM required 7-9 days for production, whereas 3-5 days of CDM production on gels were sufficient to obtain ECMs of comparable thickness. Finally, CDMs were obtained by alkaline detergent extraction, using 0.5% Triton X-100, 20 mM NH₄OH in PBS, followed by DNase I (50 U per mL) treatment. The resulting ECMs were washed three times with PBS and stored at 4° C. for up to 2 months until needed. All cell-derived ECMs underwent rigorous quality control as published (Franco-Barraza et al., Current protocols in cell biology, 2016, 71, 10 19 11-10 19 34).

### Indirect Immunofluorescence

Samples were fixed/permeabilized with 4% formaldehyde, 0.5 (v/v) % Triton X-100 and evaluated by fluorescence microscopy as previously published (Tse et al., Current protocols in cell biology, 2010, Chapter 10, Unit 10 16). For indirect immunofluorescent labeling of matrix fibers, non-extracted 3D cultures were prepared on PA gels or glass coverslips and stained with rabbit anti-mouse fibronectin antibody (25 μg/ml, Abcam, UK) followed by donkey anti-rabbit Cy5 conjugated secondary antibody (15 μg/ml, Jackson ImmunoResearch, PA). The nuclei were stained with SYBR green (Thermo Fisher Scientific, Waltham, Mass.). Images were captured using a spinning disk confocal microscope (Ultraview, Perkin-Elmer Life Sciences, Boston, Mass.) using a 60× (1.45 PlanApo TIRF) oil immersion objective. For each condition, three independent experiments were conducted and a minimum of 7 images per sample were obtained.

For quantification of the subcellular localization of ERK1/2, KRAS-PDAC cells (Franco-Barraza et al., eLife, 2017, 6) were incubated with the following primary antibodies: Rabbit anti-human phospho-p44/42 ERK1/2 (Thr202/Tyr204) (Cat No. 4370) and rabbit anti-human total—p44/42 ERK1/2 (Cat No. 9102) from Cell Signaling Technology (Danvers, Mass.) followed by the same Cy5-coupled secondary antibody detailed above. Subcellular localization of p-ERK1/2 was assessed using publicly available image analysis software, (see, world wide web at “github.com/cukie/SMIA”; SMIA-CUKIE 2.1.0) (Kaukonen et al., Nature Communications, 2016, 7, 12237). Images corresponding to the same experimental conditions and stainings were processed identically, converting 16 to 8 bit first. Next, to find the suitable thresholds that will distinguish between signal and noise needed to feed SMIA-CUKIE inputs, we proceeded to measure the histogram distribution of intensity and selected a low threshold that corresponded on average to the lower 20-to-30 percentile. Images were sorted into experimental folder batches, using the “make a batch” software (see, world wide web at “github.com/cukie/SMIA”), to include monochromatic renderings of the images of nuclei and pERK1/2 images, which then served as inputs for the SMIA-CUKIE 2.1.0 software. Mean intensity levels of pERK1/2 in nuclei vs. cytoplastic fractions were measured. Plotted results included normalized nuclei/cytoplasmic pERK1/2 intensity ratios where >1 represented higher nuclear than cytosolic activity. Representative image outputs showing pixels indicative of the above-mentioned locations were used in the figures together with input overlay images.

### Atomic Force Microscopy (AFM)-Nanoindentation

AFM-nanoindentation was carried out on a Dimension Icon AFM (BrukerNano, Santa Barbara, Calif.) using a custom-made microspherical tip. The colloidal probe was generated by attaching a 5 μm-radius polystyrene microsphere (PolySciences, Warrington, Pa.) onto the end of a tipless cantilever (Arrow-TL1Au, NanoAndMore USA, Watsonville, Calif.) using M-bond 610 epoxy (Structure Probe Inc., West Chester, Pa.). All tests were conducted in filtered 1×PBS to simulate the physiological fluidic environment. The probe tip was programmed to indent into the sample at a constant z-piezo displacement rate of 5 μm/s, up to a maximum indentation depth of about 1 μm. CDM quality control required all matrices to be at least 5 μm thick. The average ECM thickness in these experiments was about 8 μm. Each sample was tested at a minimum of 10 randomly selected locations to ensure consistency and to account for spatial heterogeneity. The indentation modulus E_{ind} was calculated by fitting the loading portion of each indentation force-depth curve to the Hertz model

$$F = \frac{4}{3} \frac{{E}_{ind}}{( 1 - {v}^{2} )} {R}_{tip}^{1 / 2} {D}^{3 / 2} , \qquad ( 1 )$$

where F is the indentation force, D is the indentation depth, v is the poisson's ratio (0.49 for highly swollen hydrogels) (Zhang et al., Science Signaling, 2014, 7, ra42), and Rₜᵢₚ is the radius of the probe tip (about 5 μm). Since the thickness of the PA gels (>200 μm) is orders of magnitude greater than the maximum indentation depth, the substrate constraint effect was minimal, and thus, finite thickness correction was not needed. The comparison between CDMs and adjacent bare gels was carried out by probing regions with or without the ECM on the same gel for consistency.

### Mathematical Model for Predicting Cell Shape

Cells change their shapes with respect to the properties of the underlying substrate. To understand how substrate stiffness influences cell morphology, we considered a cell cultured on a 2D substrate. We used an energy criterion to determine the cell shape, i.e. we hypothesized that a cell adjusts its shape in order to minimize the total free energy of the cell-substrate system. The total free energy can be written as,

E=E_{cell}+Eₘₐₜᵣᵢₓ+Eᵢₙₜ (2),

where E_{cell} is the cell energy, Eₘₐₜᵣᵢₓ is the elastic energy of the ECM and Eᵢₙₜ is the interface energy (including the basolateral cell-substrate interface and the apical free cell surface). The cell energy is a function of elastic energy (accounting for cell deformation) and the motor density (accounting for contractility). Based on the model for contractile cells (Shenoy et al., Interface focus, 2016, 6, 20150067), it can be written as,

E_{cell}=∫_{Cell}U_{C}(εᵢⱼ^{C},ρᵢⱼ)dV (3)

where U_{C} is the cell energy density, εᵢⱼ^{C} is the elastic deformation of the cell, and ρᵢⱼ is the motor density. The interface energy consists of the basolateral cell-substrate interface energy and the apical free cell surface energy:

Eᵢₙₜ=γ_{CM}S_{CM}+γ_{C}S_{C} (4),

where γ_{CM} and γ_{C} are interface/surface energy density for cell-matrix interface and free cell surface respectively, S_{CM} and S_{C} are the area for cell-substrate interface and free cell surface respectively.

We characterized the cell shape by defining the aspect ratio f=a/c. For a given substrate (a fixed stiffness), we computed the total free energy of the cell-substrate system for various aspect ratios, and chose the energy minimized one as the preferred cell shape. Next, we varied the substrate stiffness and obtained the cell aspect ratio as a function of stiffness.

### Mathematical Model for Contractile Cells Indicative of Fiber Alignment

In terms of the stress-dependent regulation of cell contractility, the contractile stress of the actin network can be written (Shenoy et al., Interface focus, 2016, 6, 20150067) as,

σ=ρ+Kε (5),

where ρ is the density of force-diples (representing myosin motors/contractility) in the actin network, ε is the strain of the actin network, and K is the effective passive stiffness of the actin network. The contractility itself depends on the mechano-chemical coupling through the signaling pathways discussed above;

$$ρ = \frac{β {ρ}_{0}}{β - α} + \frac{α κ - 1}{β - α} ɛ , \qquad ( 6 )$$

where ρ₀ is the contractility in the absence of adhesions, α and β denote mechano-chemical coupling parameters. Additional details of this model have been described elsewhere (Shenoy et al., Interface focus, 2016, 6, 20150067).
Short Interfering RNA (siRNA) Transfections

Transient transfections were performed on hTERT/E6/E7/KRAS (KRAS) cells using Lipofectamine® 2000 and following manufacturer's instructions (Thermo Fisher Scientific, Waltham, Mass.). Non-targeting SMARTpool and siRNA targeting ERK1 or ERK2, each comprising four distinct siRNA species, were obtained from Life Technologies-Dharmacon (Lafayette, Colo.). KRAS cells, 1×10⁵ per well in a 6-well plate were used. Transfections were carried out in basal medium without FBS and antibiotics. Cells were trypsinized, counted and mixed with transfection medium as per manufacturer's instructions for 5 hours, after which media was replaced with regular media for an additional 48 hours. In some experiments the cells were trypsinized 24 hours post transfection and used for spheroid formation followed by spheroid spread assay (see below).

### Western Blotting

At the end of the experiments, KRAS cells were lysed with cell lysis buffer from Cell Signaling Technology (Danvers, Mass.) supplemented with Pierce™ Phosphatase and protease Inhibitor Mini Tablets (Cat Nos. 88667 and 88665, respectively) from Thermo Fisher Scientific (Waltham, Mass.). Proteins were SDS-PAGE dissolved and transferred to PVDF membranes. Blots were incubated with the following primary antibodies: Rabbit anti-human Phospho-p44/42 ERK1/2 (Thr²⁰²/Tyr²⁰⁴)(Cat No. 4370) and rabbit anti-human total-p44/42 ERK1/2 (Cat No. 9102) obtained from Cell Signaling Technology (Danvers, Mass.). Anti-phospho-p90RSK1 (Ser³⁸⁰) (Cat No. 04-418) and anti-human glyceraldehyde 3-phosphate dehydrogenase (GAPDH) (Cat No. MAB374) obtained from Millipore (Billerica, Mass.). Horseradish peroxidase-conjugated, anti-species matched, secondary antibodies were obtained from Sigma Aldrich (St. Louis, Mo.). Protein bands were visualized using the Protein Simple FluorChemE System, (San Jose, Calif.). Nuclear levels of pERK1/2 were assessed using the Subcellular Protein Fractionation Kit (Thermo Scientific, Waltham, Mass.). Isolation of nuclear versus whole cell fractions was carried out according to the manufacturer's instructions.

### Ki67 Cell Proliferation Assay

Pancreatic human epithelial cells (hTERT and KRAS) were plated at a density of 2×10⁴ cells/mL on the above mentioned CDM on gels, or coverslip control samples and incubated for 24 hours. Cells were fixed prior to staining with anti-Ki67 antibody (Cat No. ab15580) using our previous protocols. The fraction of proliferating cells was measured by counting the number of cells stained positive for Ki67 divided by total number of nuclei stained using Hoechst 33342 solution (Calbiochem, Billerica, Mass.). At least 5 images were taken per condition and the experiment was performed. Data was pooled from all three experiments and plotted.

### Spheroid Spreading Assay

Red fluorescence protein (RFP)-expressing KRAS cells generated in our laboratory were trypsinized and suspended in spheroid formation media (Irvine Scientific, Santa Ana, Calif., Catalog ID: 91130) and allowed to incubate overnight. Thirty μl drops containing 2.5×10³ cells were carefully placed on a lid of a sterile petri dish. The dish was filled with 5 mL media and the lid with the “hanging drops” was carefully placed, drops face down, and incubated overnight. Spheroids were removed from the lids, one by one, placed onto the diverse substrates or CDMs, and allowed to adhere for 2 hours. Subsequently, the spheroid formation media was diluted with regular pancreatic epithelial growth media (see above). Following a 48-hour incubation, cell spreading was visualized using an inverted fluorescence microscope equipped with epifluorescent image acquisition capabilities. Data were normalized to the initial size of each spheroid at time 0 hour. When indicated, spheroids were treated overnight with 20 μM of the MEK1/2 inhibitor U0126 from Calbiochem or DMSO, while siRNA transfection. The images were processed using MetaMorph 7.8.1.0 software (Molecular Devices, Downingtown, Pa.). A minimum of 5 spheres per condition were analyzed in at least three independent experiments.

### Statistical Analysis

Experiments were performed at least in duplicates and repeated independently at least three times. Data was plotted using GraphPad Prism and analyzed using unpaired Student's t-test. Values were plotted as median ±interquartile range or mean±standard deviation as indicated in the corresponding figure legends. Asterisks depicting statistical significance are provided in the assorted figures and tables and described in the legends.

### Example 2: CDM/Gel System Characterizations

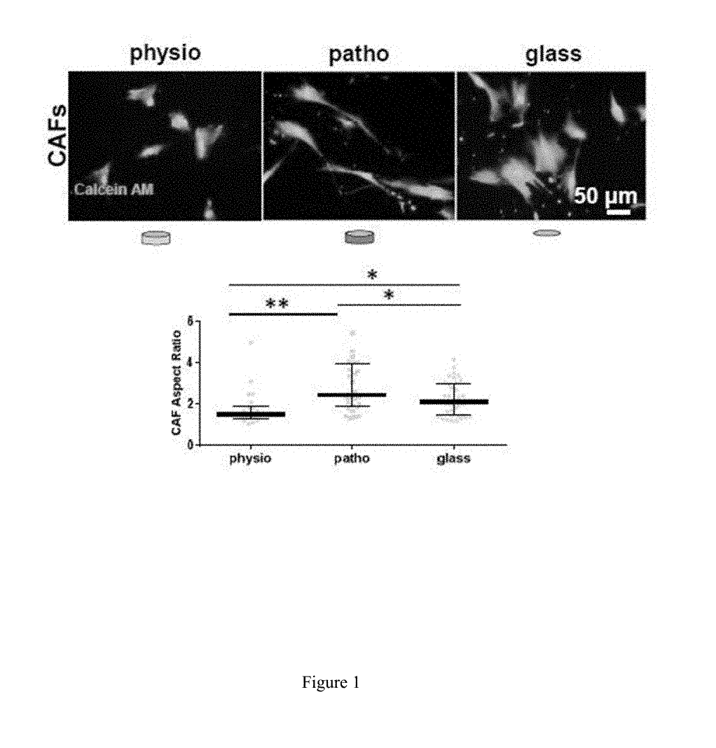

To assess the changes in CAF morphology in response to stiffness, CAFs were cultured overnight onto physiological (physio) or pathological (patho) stiffness PA gels (Itoh et al., JMRI, 2016, 43, 384-390) and their maximum length/breadth (cell aspect ratio) were measured. Previous studies have documented that activated CAFs depict spindled shape morphology and contain high levels of alpha-smooth muscle actin (Kalluri, Nat Rev Cancer, 2016, 16, 582-598; Ronnov-Jessen et al., Journal of clinical investigation, 1995, 95, 859-873). We observed that CAFs display highly elongated/spindled morphology on patho stiffness gels or coverslip; however, on physio PA gel stiffness, CAFs failed to elongate and displayed a rounded morphology (see, FIG. 1). Interestingly, a biphasic tendency of change in CAF morphology was observed, where CAFs seemed to show maximum levels of cell elongation at intermediate stiffness (about 7 kPA), but not extremely stiff substrate (coverslip) (see, FIG. 1). Our data agrees with other studies that show a biphasic response of cell polarization and/or cell motility as a function of substrate stiffness (Peyton et al., Journal of cellular physiology, 2005, 204, 198-209; Pathak et al., Proc. Natl Acad. Sci. USA, 2012, 109, 0334-10339; and Lang et al., Acta biomaterialia, 2015, 13, 61-67).

Referring specifically to FIG. 1, changes in substrate stiffness that dictate a biphasic distribution of CAF aspect ratios are shown. Epi-fluorescent microscopy images depict cell shape (Calcein AM; green) and nuclei (blue) of CAFs cultured onto physio (about 1.5 kPa), patho (about 7 kPa) PA gels and coverslip. A corresponding graph presenting CAF aspect ratios (length/breadth) calculated using MetaMorph software is shown below. Data is presented as median ±interquartile range.

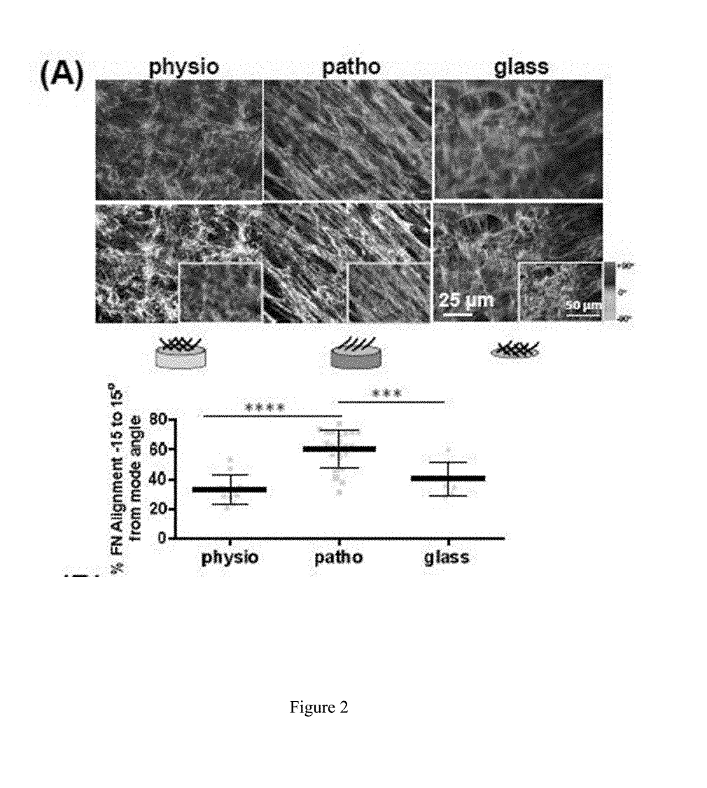

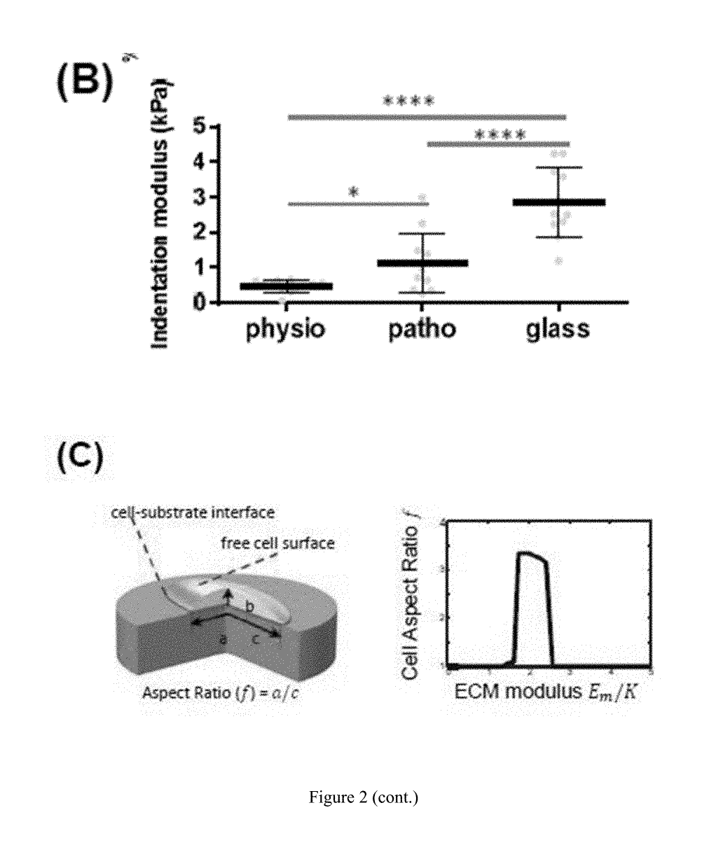

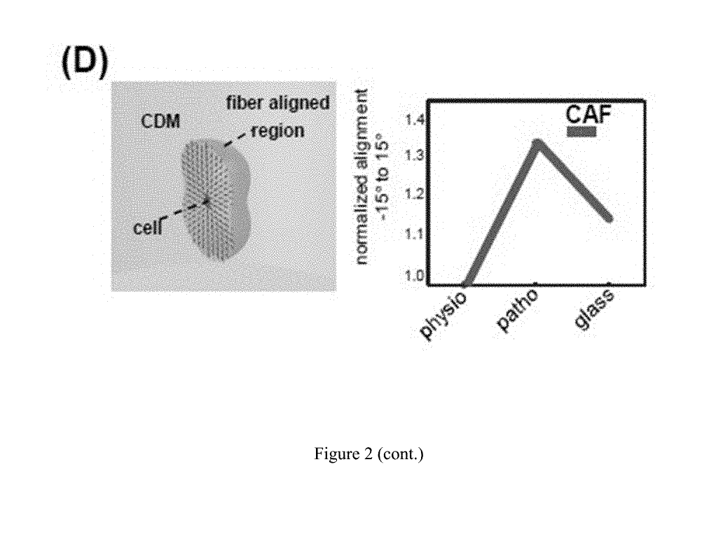

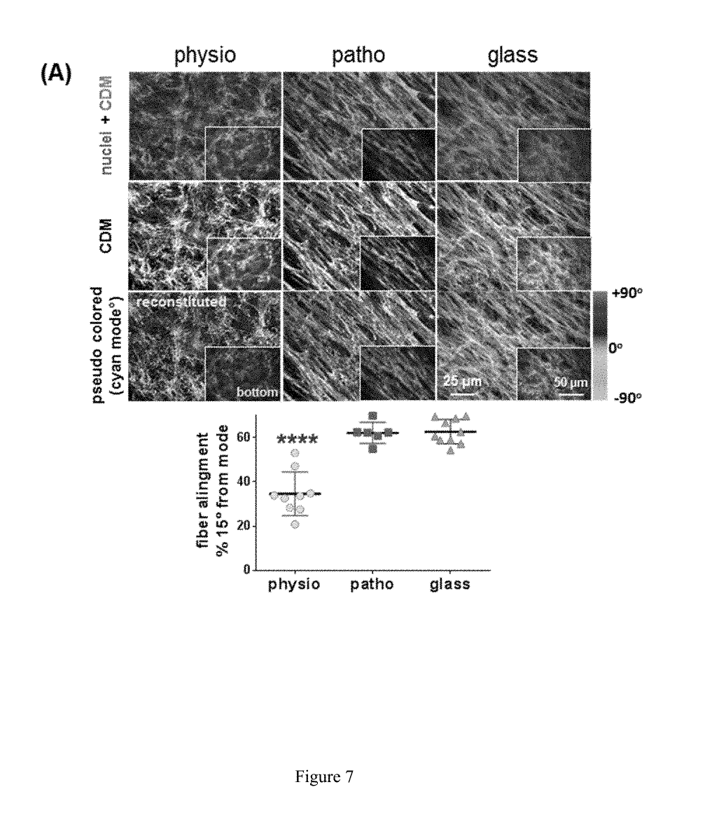

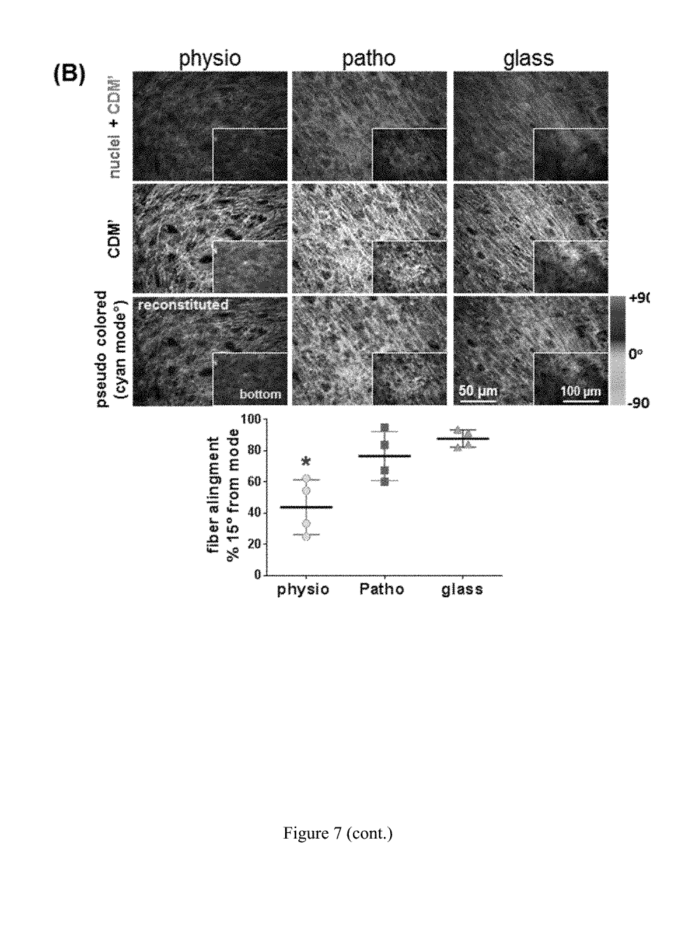

Next, we tested whether there is a positive correlation between stiffness-dependent changes in CAF cell-aspect ratio and CAF-derived ECM alignment/anisotropy. On glass, CAFs typically form a parallel aligned matrix, whereas normal fibroblasts produce a randomly organized matrix (Franco-Barraza et al., Current protocols in cell biology, 2016, 71, 10 19 11-10 19 34; and Franco-Barraza et al., eLife, 2017, 6). We tested whether changing underlying rigidity via culturing CAFs on physio gel stiffness no longer produces parallel aligned fibers of CAF-derived matrix. For this, CAFs were cultured onto the above-mentioned Collagen I coated physio and patho gels and prompted to produce ECMs onto them. The influences of substrate stiffness on the resulting anisotropic levels of CAF-derived ECM was measured by accounting for the region where the fibers would align, ranging from −15° to 15° towards the long axis of the cell. Our data suggests that producing matrices from CAFs on physio polyacrylamide gels resulted in prominent decreases in CAF-derived ECM anisotropy. Noticeably, results clearly rendered the predictive biphasic ECM anisotropy where anisotropy levels first increased with stiffness, then decreased for CDMs produced onto the very stiff substrate (i.e., coverslip condition), suggesting that there is a positive correlation among cell aspect ratio and CAF-CDM anisotropy as a function of stiffness. Interestingly, we also observed that CDMs on gels require fewer number of days for production (3-5 days on gels, as opposed to 7-9 days on glass to achieve same μm of thickness (Franco-Barraza et al., Current protocols in cell biology, 2016, 71, 10 19 11-10 19 34)). As evident in FIG. 2 (Panel A), matrix produced by CAFs on glass is unorganized on day 3 of production (since the matrix is too thin and not fully formed), however after 7-9 days of production, CAF-ECM on glass becomes organized as described in our previously published protocols (Franco-Barraza et al., Current protocols in cell biology, 2016, 71, 10 19 11-10 19 34) (see, FIG. 7). Interestingly, the observed heterogeneity amongst top versus bottom layers of fibronectin fibers and nuclear orientation seemed to significantly reduce after the gels were incorporating in the CDMs (see, FIG. 7). This may suggest that gels may be acting as “cushions” between coverslip and the top surface of the gel, thus preventing CAFs from sensing the artificial stiffness of underlying coverslip.

Referring specifically to FIG. 2 (Panels A-D), changes in substrate stiffness that dictate a biphasic distribution of fibroblastic derived ECM alignment which in turn is dictated by their biphasic cell aspect ratios are shown. Panel A shows reconstituted maximum projections of confocal images obtained from CDM produced by CAFs cultured on physio and patho substrates (top). Staining of fibronectin (green) and nuclei (blue) and monochromatic images are shown. Colors in bottom panels depict angle distributions of CDMs, obtained using ‘OrientationJ’ plugin of Image-J software. Images were normalized using hue values for common/mode, cyan, and angle visualization as indicated by the bar on the right. Note that the ECM fiber anisotropy is at peak levels when the matrix is produced on stiff (about 7 kPa gel) gels. A graph (below) shows percentages of fibers distributed at 15° angles from the mode for the indicated experimental conditions. Panel B shows indentation moduli of decellularized CDMs produced by CAFs cultured onto the indicated substrates. Results are presented as mean±standard deviation. Asterisks denote the following order of significance: * p<0.05, ** p<0.01, *** p<0.005 and **** p<0.001. Panel C shows a mathematical model to validate biphasic distribution in CAF shape as a function of stiffness. A schematic scheme of a cell (green) spread onto a 2D substrate (blue) (right) is shown. To calculate interface energy contribution to total energy, both cell-substrate interface (red) and free cell surface (gold) are modeled as isotropic surfaces with surface energy γ_{CM} and γ_{C} (γ_{C}>γ_{CM}), respectively. The minimum shape energies show biphasic response to changes in substrate modulus. K is the effective passive stiffness of the cellular actin network. Panel D shows a mathematical model to validate that anisotropy levels of CAF-derived CDMs are dictated by the biphasic cell's aspect ratios. A schematic depiction of the model suggests that fiber alignment induced by single cell contraction (left): Light red area shows the spread of ECM that is affected by a single cell, shown in the center. Red cones indicate predicted local fiber orientations. A graph presenting predicted fibronectin alignment using the above model is shown.

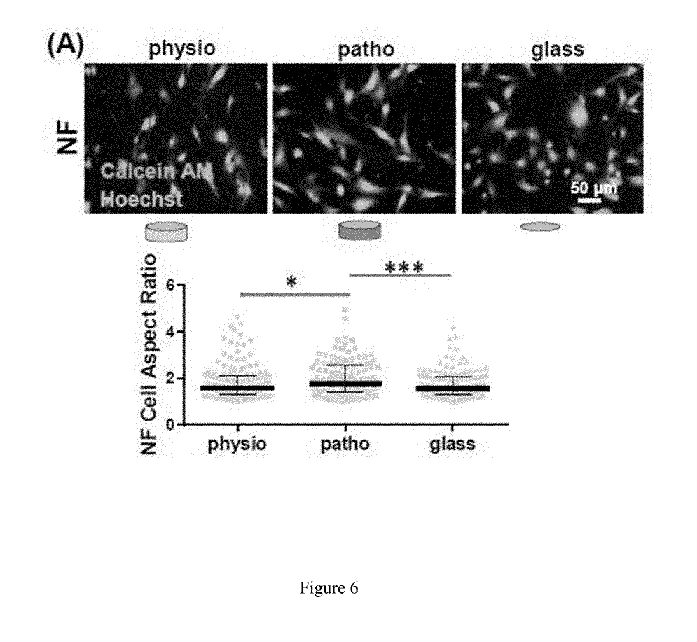

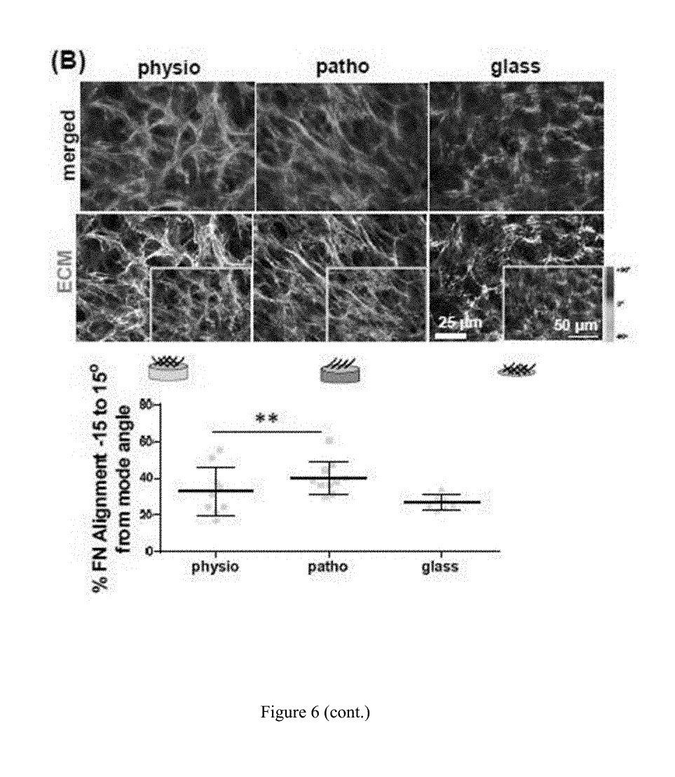

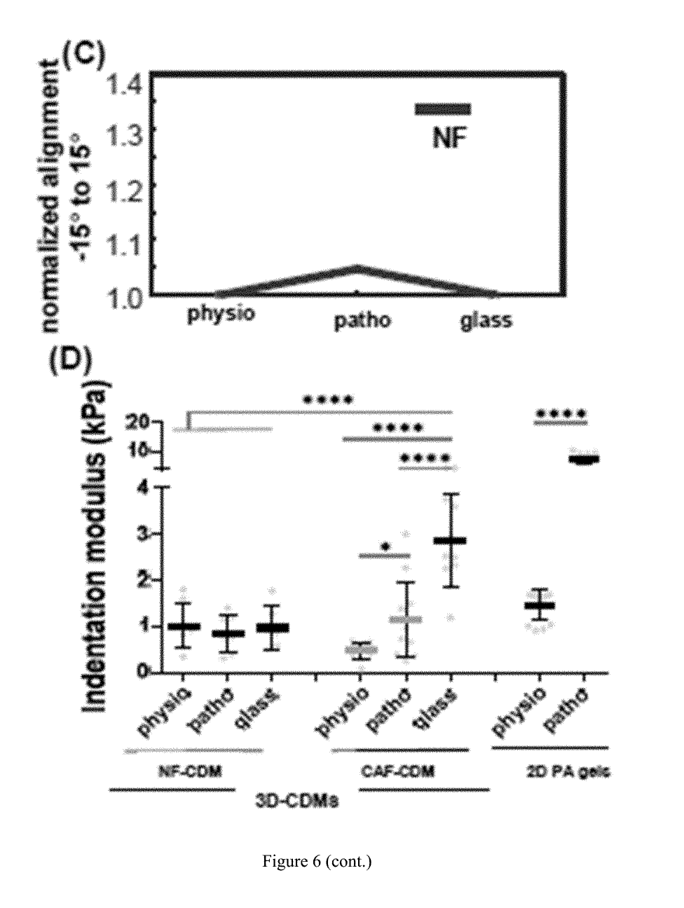

Referring specifically to FIG. 6 (Panels A-D), changes in substrate stiffness dictate a biphasic distribution of NF-derived ECM alignment which in turn is dictated by their biphasic cell aspect ratios. Panel A shows changes in substrate stiffness dictate a biphasic distribution of CAF aspect ratios. Epi-fluorescent microscopy images depicting cell shape (Calcein AM; green) and nuclei (blue) of CAFs cultured onto physio (about 1.5 kPa) or patho (about 7 kPa) PA gels and coverslip (top). Graph presenting CAF aspect ratios (length/breadth) calculated using MetaMorph software (below). Data is presented as median ±interquartile range. Panel B shows reconstituted maximum projections of confocal images obtained from CDM produced by NFs cultured on physio and patho substrates (top). Staining of fibronectin (green) and nuclei (blue) and monochromatic images are shown. Colors in bottom panels depict angle distributions of CDMs, obtained using ‘OrientationJ’ plugin of Image-J software. Images were normalized using hue values for common/mode, cyan, and angle visualization as indicated by the bar on the right. Note that the ECM fiber anisotropy is at peak levels when the matrix is produced on stiff (about 7 kPa gel) gels. The graph (below) shows percentages of fibers distributed at 15° angles from the mode for the indicated experimental conditions. Panel B shows mathematical model to validate biphasic distribution in CAF shape as a function of stiffness. Panel C shows mathematical model to validate that anisotropy levels of CAF-derived CDMs are dictated by the biphasic cell's aspect ratios. Panel D, indentation moduli of decellularized CDMs produced by NFs or CAFs cultured onto the indicated substrates. Results are presented as mean±standard deviation. Asterisks denote the following order of significance: * p<0.05, ** p<0.01, *** p<0.005 and **** p<0.001.

Referring specifically to FIG. 7, heterogeneity in fibronectin fibers amongst top versus bottom layers is minimized when CAF-CDM is derived on gels instead of glass is shown. Similar results were obtained using additional patient-harvested CAFs (see, FIG. 7, panel B). Reconstituted confocal microscopy images obtained from CDMs produced by CAFs onto physio or patho stiffness polyacrylamide gels as well as glass coverslip is shown. Images are shown for 3 days of production of CAF-CDM on gels whereas 8 days for production for CAF-CDM on glass. Staining of fibronectin (green) and nuclei (blue) and individual monochromatic images are shown. Colors in the bottom third row panel depict angle distributions of CDMs, obtained using ‘OrientationJ’ plugin of Image-J software. Images were normalized using hue values for common/mode, cyan, and angle visualization as indicated by the bar on the right. Inserts represent bottom layers and are provided to show that CDMs generated onto gels are homogenic, while glass generated CDMs are heterogeneous.

To question whether CAF-derived CDMs with similar anisotropic fiber orientation levels also share similar stiffness, the indentation modulus of the CDMs generated on physio or patho stiffness substrates were measured using atomic force microscopy (AFM). We observed that CAF-generated ECM on physio substrate showed approximately two-fold decrease in matrix indentation modulus. Unlike ECM anisotropy and cell-aspect ratio, the changes in indentation modulus were not biphasic. The data suggests that generating CAF-derived ECMs on physiological substrate stiffness can result in normalization of key mechanical properties of desmoplastic CAF-CDM remodeling such as ECM alignment and ECM indentation modulus.

We incorporated two mathematical models to explain the observed biphasic distribution in CAF cell aspect ratio (contractile cell model) and CAF-derived fiber alignment (fibrous ECM model) as a function of stiffness. Previous studies describe a “contractile cell model” to explain correlation between stiffness and cell polarization. In this model, as cells sense environments of different stiffness, variations in their chemical energy (arising due to engagement of myosin motors) and mechanical energy (due to stiffness) takes place which prompts the cells to assume a shape which is most energetically favorable. Here, we used a similar energy criterion to determine CAF shape on physio or patho stiffness polyacrylamide gels or coverslip. The model predicts that the cell aspect ratio shows a biphasic response to the ECM stiffness increase (see, FIG. 2, Panel C), which is in consistent with our experimental results (see, FIG. 1). Based on the shapes of the cells achieved, we classified CAF shape on physio, patho and coverslip 2D as hemispherical (half elliptical), half-cigar (elliptical), and pancake (circular) shape. The image in FIG. 2, Panel C is representative of a cell on surface of a 2D substrate where cell aspect ratio (f) is defined as f=a/c, where a is length and c is breadth of the cell. The blue line in the adjacent graph depicts biphasic changes in cell-aspect ratio of CAFs, where maximum CAF elongation is achieved at intermediate stiffness (2 kPA). It is important to note that while both model and our experimental results predict a biphasic mode of fibroblast shape changes, differences were evident with regards to the stiffness values that accounted for the highest cell elongations (2 kPa were predicted by the model while about 7 kPa showed an elongation peak experimentally). The results validate our experimental observations, suggesting that culturing CAFs on physio gel results in normalization of activated CAF morphology, which follows a biphasic distribution.

We designed a second mathematical model that uses the experimental cell aspect ratios from FIG. 1 to predict CAF-derived CDM fiber alignment. This model is based on a recently published, discrete fiber network-inspired, “constitutive material model (or fibrous ECM model)” (Wang et al., Biophysical journal, 2014, 107, 2592-2603), which addresses fiber alignment as well as long-range force transmission (ability of cells to sense each other at long distance in fibrous ECM). By integrating the fibrous ECM model and contractile cell models (based on the measured aspect ratios), we were able to simulate the active crosstalk between CAFs and their CDM and predict the influence of cell contraction on the local/initial fiber alignment. Our model predicted that cell contraction by CAFs prompts fiber alignment in a relative large region, which is approximately 300 times of that of the cell volume. Both our model predictions and experimental results demonstrate that CAFs become most elongated when cultured onto a surface of intermediate stiffness, indicating that the cellular contraction will be most uniaxial when CAFs are cultured onto substrate of intermediate stiffness. Hence, the anisotropy levels of CDM fibers are most pronounced for the intermediate stiff matrices. On the contrary, CAFs cultured on physio stiffness substrate were less elongated, therefore they exerted lesser degree of contractions onto the matrix, resulting in formation of less organized matrix. FIG. 2, Panel D depicts a schematic of a cell (black) in the center of ECM (light red background), where the red cones indicate the predicted fiber orientation. The blue line in graph in FIG. 2, Panel D clearly shows a biphasic distribution in ECM anisotropy which positively correlates with biphasic distribution in CAF shape. Taken together, both our experimental findings and mathematical model suggests that substrate stiffness dictates a biphasic change in CAF fibroblastic shape, which in turn dictates the anisotropy levels of fibroblastic CDM fiber.

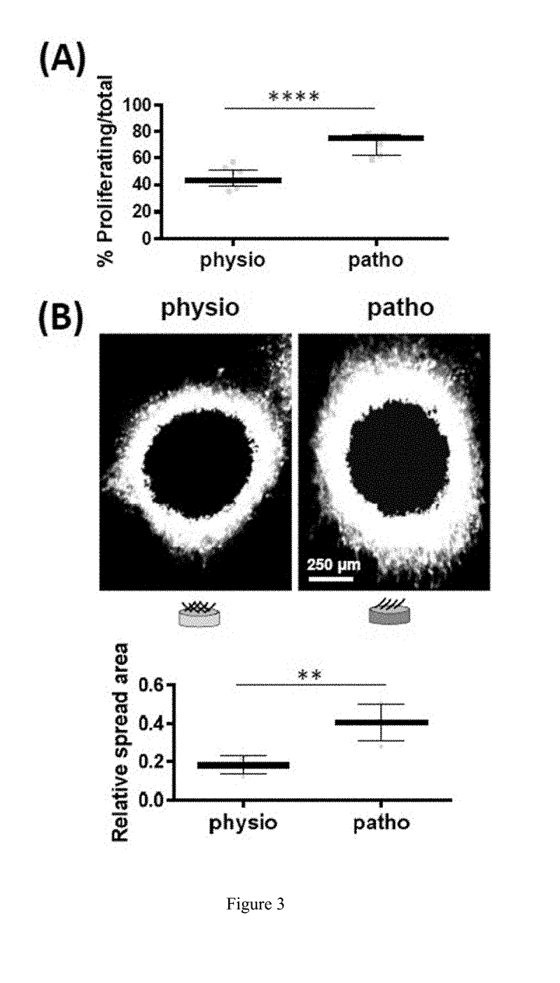

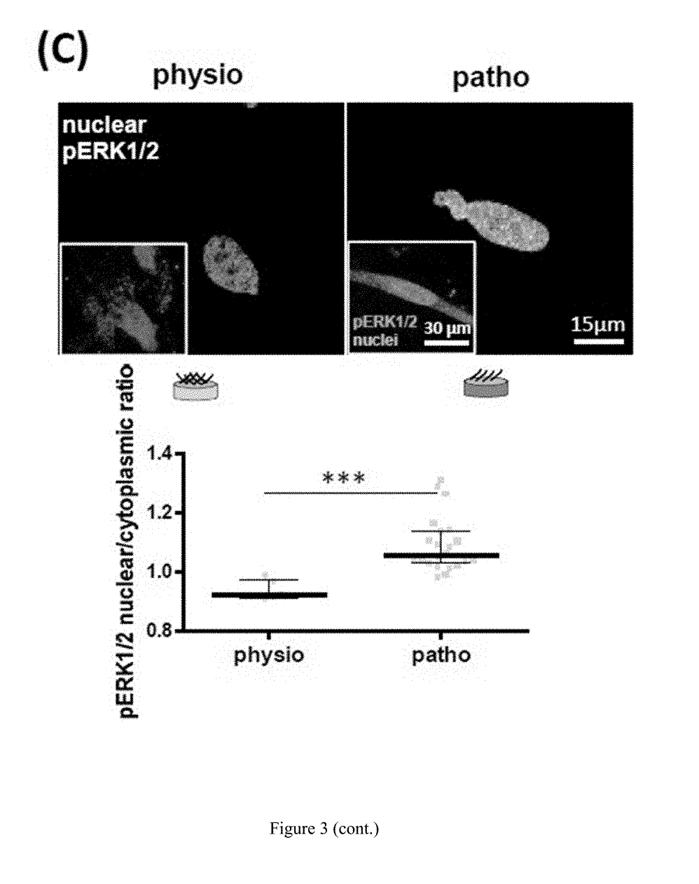

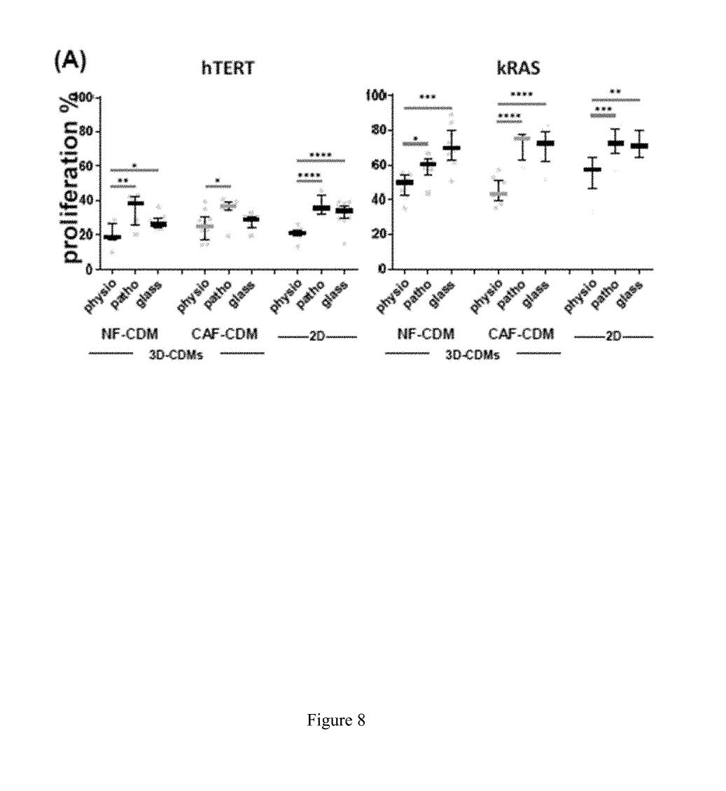

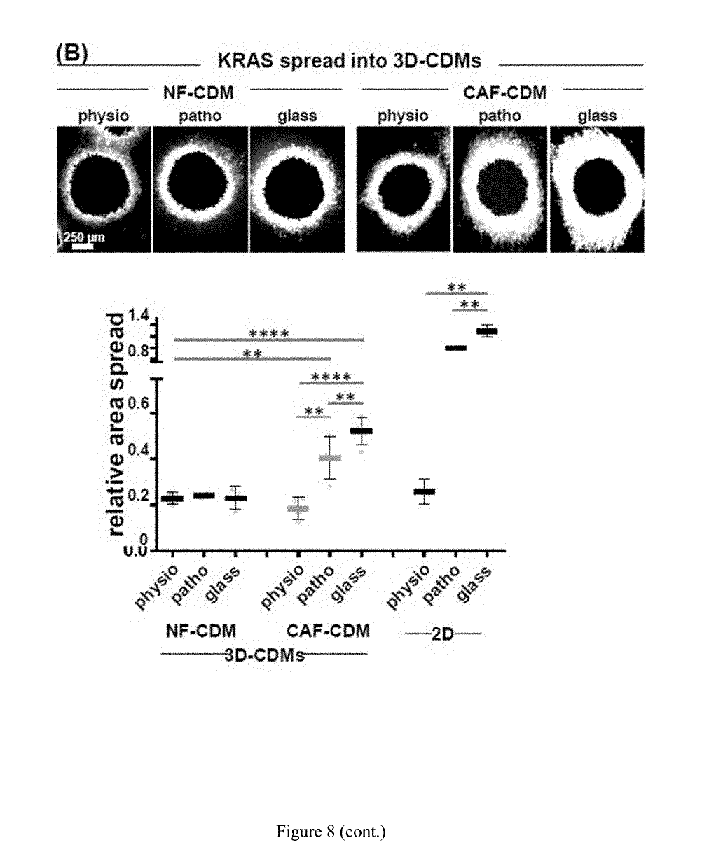

Studies have demonstrated that fibroblastic CDMs, produced by CAFs, impart a tumor permissive phenotype upon cancer cells, whereas ECMs obtained by normal fibroblasts (NFs) present tumor suppressive capabilities. Here, we investigated whether CAF-derived ECM on physio stiffness polyacrylamide gel could restrict tumorigenic behavior such as proliferation and migration within pancreatic cancer cells. For this analysis, CAFs were extracted from the original CAF-derived matrices using ammonia/triton as described in our previous protocols (Franco-Barraza et al., Current protocols in cell biology, 2016, 71, 10 19 11-10 19) and the decellularized CAF-derived matrices were used as substrates for re-plating pancreatic cancer cells. Previously described and well-characterized KRAS mutated human pancreatic ductal epithelial cell were used for replating within the decellularized CAF-ECMs (Campbell et al., 2007, Cancer research, 67, 2098-2106). We started by determining whether the CDMs generated on physio gel could restrict proliferation in pancreatic cancer cells. For this, KRAS mutated pancreatic cancer cells were cultured within the assorted CDMs and proliferation rates were measured via Ki67 scoring. Benign (hTERT immortalized) isogenic pancreatic cancer cells were treated as controls (see, FIG. 8, Panels A and B). We observed that CDMs produced by CAFs on physio gels lead to significant decreases in growth in KRAS cells (see, FIG. 3, Panel A). Next, we proceeded to test whether CAF-derived ECM on physio gels restricted migration in KRAS cells. For this, red fluorescent protein (RFP) transfected KRAS cell spheroids were generated and seeded within the assorted matrices and their relative area spreads were recorded at various time points using epifluorescence microscopy. The spheroid spread areas were quantified using the MetaMorph software. On CAF-CDM generated on physio gels, we observed a dramatic decrease in kRAS cell spheroid spreading compared to those generated on patho gels (see, FIG. 3, Panel B) or glass controls (see, FIG. 8, Panels A and B). The results suggest that biomechanical manipulation of CAF-ECM via changing substrate rigidity can reverse tumor promoting features of CAF-ECMs such as cell proliferation and migration. Upon comparing all conditions (including data obtained from control fibroblasts in FIG. 8, Panels A and B, we conclude that stiffness of 3D CDMs does not correlate with observed changes in either proliferation or invasion, however matrix alignment was a major predictor of tumorigenic responses in KRAS cells.

Referring specifically to FIG. 3 (Panels A-C), CAF-derived CDM generated on physiological substrates restricts tumorigenic responses in kRAS cells via loss of nuclear pERK1/2 is shown, Panel A shows a Ki67 proliferation assay depicting levels of proliferating kRAS cells on physio or patho stiffness substrates. Levels were assessed via indirect immunofluorescence using nuclei stain counts for normalization purposes and results (median ±interquartile range) were plotted. Panel B shows epifluorescence microscopy images showing spread of RFP expressing KRAS spheroids within CDMs generated on physio versus patho stiffness substrates. Spheroids of known even sizes, were allowed to spread for 48 hours. A graph below displays 95 percentile fluorescence intensities were used to measure area spreads. Panel C shows indirect immunofluorescence was conducted using anti-pERK1/2 antibody (shown in green) and cell nuclei were labeled using SYBR green (shown in blue). Monochromatic images were provided as inputs and relative percentages of nuclear pERK1/2 values as well as image outputs, depicting pERK1/2 cytosolic vs. nuclear localization distributions, were obtained using SMIA-CUKIE 2.1.0 (see, world wide web at “github.com/cukie/SMIA”). KRAS cells were cultured overnight within assorted CDMs. Asterisks denote the following order of significance: * p<0.05, ** p<0.01, *** p<0.005 and **** p<0.001.

Referring specifically to FIG. 8 (Panels A-B), CAF-derived matrices generated on physio gels restrict tumorigenic responses in both kRAS mutated cells and benign pancreatic cancer cells. Panel A shows syngeneic human pancreatic epithelial (hTERT) and cancer (KRAS) cells were cultured within the assorted 3D extracted CDMs or onto 2D substrate controls for 24 hours. Ki67 levels were assessed via indirect immunofluorescence using nuclei stain counts for normalization purposes and results (median ±interquartile range) were plotted. Panel B shows RFP expressing KRAS spheroids, of known even sizes, were allowed to spread on assorted CDMs for 48 hours. Images were acquired at times 0 and 48 hours and 95 percentile fluorescence intensities were used to measure area spreads, as shown in the three images on the bottom left column. The top row shows representative experimental images indicating the relative masked areas that were used to measure cell spread, which were plotted on the bottom right graph. Asterisks denote the following order of significance: * p<0.05, ** p<0.01, *** p<0.005 and **** p<0.001.

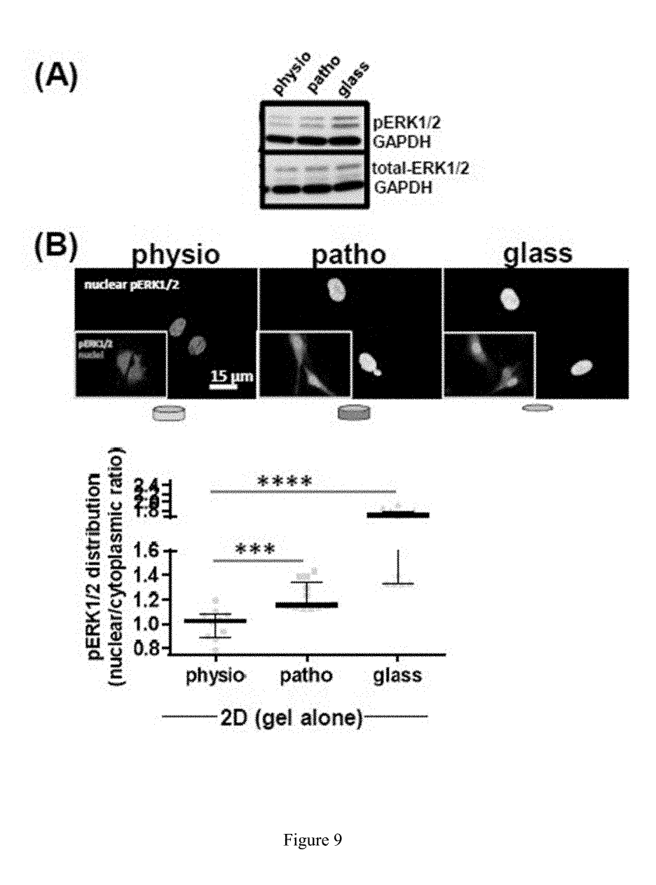

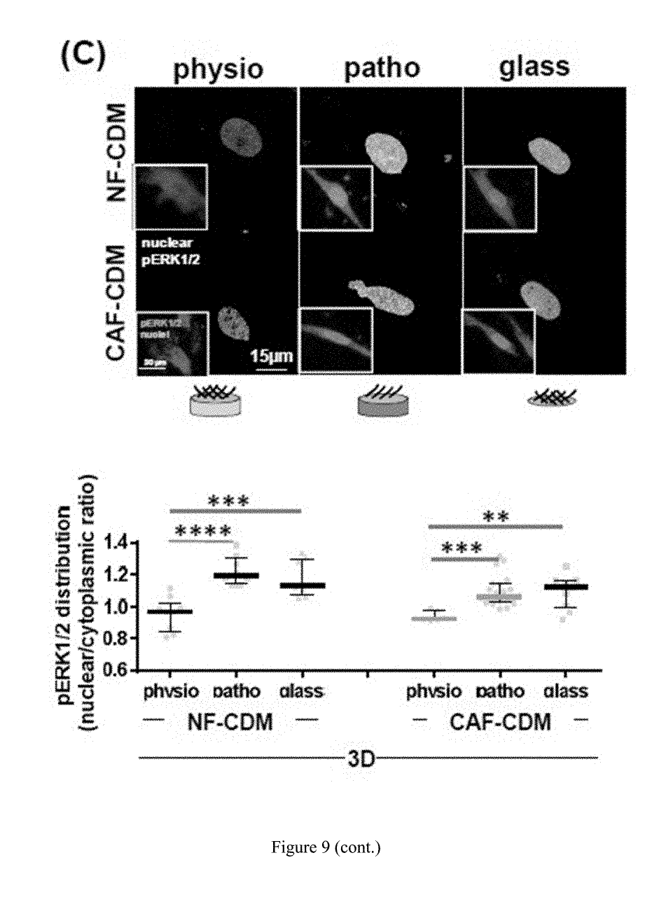

We next proceeded to query the mechanism of tumor restriction cells mediated by physio CAF-CDMs. Levels of phosphorylated extracellular regulated kinase-1/2 (pERK1/2), known to act downstream to activated KRAS were measured. We observed that stiffness alone did not seem to alter pERK1/2 levels in KRAS cells cultured on physio versus patho gels (see, FIG. 9, Panel A). Therefore, we looked for differences in pERK1/2 localization. Many cancers, including pancreatic cancer display high levels of nuclear pERK1/2 localization. Confocal microscopy images, corresponding to maximum reconstituted projections of pERK1/2 positive pixels were stratified according to nuclei positive or negative pixel locations and quantified using the simultaneous multispectral imaging analysis software, SMIA-CUKIE. Significant differences in the subcellular localization of pERK1/2 were evident showing a linear tendency for enriched nuclear pERK1/2 localization as 2D stiffness was increased (see, FIG. 9, Panel B and Table 2). Using these observations as controls, we next tested whether significant differences in the subcellular localization of pERK1/2 were also evident in KRAS cells cultured within CDMs generated on physio versus patho stiffness gels. We demonstrated that CAF-generated ECMs produced on physio substrate stiffness showed loss of pERK1/2 in the nuclei of KRAS cells whereas high levels of nuclear pERK1/2 continued to remain for cells cultured on patho stiffness substrate (see, FIG. 3, Panel C). The results suggest that loss of nuclear activity of pERK1/2 is linked with tumor restriction responses in kRAS associated pancreatic cancer.

Referring specifically to FIG. 9 (Panels A-C), nuclear localization, but not total protein levels of activated ERK1/2 are altered in kRAS cells cultured on physio versus patho substrate stiffness gels are shown. Panel A shows a Western blot depicting pERK1/2 and total ERK1/2 levels in kRAS cells cultured on physio (about 1.5 kPa) or patho (about 7.5 kPa) stiffness bare gels and onto glass coverslip after overnight incubation. Panel B shows indirect immunofluorescence microscopy images of KRAS cells cultured on physio, patho, and coverslip 2D substrates following overnight incubation. Panel C shows kRAS cells were cultured overnight within assorted CDMs. Cells were stained with pERK1/2 antibody (green) and their nuclei was counterstained using SYBR green (blue). Monochromatic images were provided as inputs and relative percentages of nuclear pERK1/2 values as well as image outputs, depicting pERK1/2 nuclear localization distributions. Asterisks denote the following order of significance: * p<0.05, ** p<0.01, *** p<0.005 and **** p<0.001.

TABLE 1
Nanoindentation moduli of assorted CDMs and gel substrates

| Assorted CDMs or substrate | P |
|---|---|
| comparisons | values |
| physio vs patho | **** |
| physio vs CAF-CDM physio | **** |
| physio vs CAF-CDM patho | p = 0.20 |
| physio vs CAF-CDM glass | **** |
| physio vs NF-CDM physio | * |
| physio vs NF-CDM patho | p = 0.70 |
| physio vs NF-CDM glass | * |
| patho vs CAF-CDM physio | **** |
| patho vs CAF-CDM patho | **** |
| patho vs CAF-CDM glass | **** |
| patho vs NF-CDM physio | **** |
| patho vs NF-CDM patho | **** |
| patho vs NF-CDM glass | **** |
| CAF-CDM physio vs CAF- | * |
| CDM patho | |
| CAF-CDM physio vs CAF- | **** |
| CDM glass | |
| CAF-CDM physio vs NF-CDM | ** |
| physio | |
| CAF-CDM physio vs NF-CDM | p = 0.10 |
| patho | |
| CAF-CDM physio vs NF-CDM | ** |
| glass | |
| CAF-CDM patho vs CAF-CDM | *** |
| glass | |
| CAF-CDM patho vs NF-CDM | p = 0.67 |
| physio | |
| CAF-CDM patho vs NF-CDM | p = 0.44 |
| patho | |
| CAF-CDM patho vs NF-CDM | p = 0.65 |
| glass | |
| CAF-CDM glass vs NF-CDM | *** |
| physio | |
| CAF-CDM glass vs NF-CDM | p = 0.22 |
| patho | |
| CAF-CDM glass vs NF-CDM | *** |
| glass | |
| NF-CDM physio vs NF-CDM | p = 0.43 |
| patho | |
| NF-CDM physio vs NF-CDM | p = 0.91 |
| glass | |
| NF-CDM patho vs NF-CDM | p = 0.48 |
| glass | |

p-value table indicative of statistics obtained during indentation moduli measurements of decellularized CDMs generated by NFs or CAFs onto the assorted substrates using bare gels (physio and patho) as controls. Asterisks denote the following order of significance: * p<0.05, ** p<0.01, *** p<0.005 and **** p<0.001.

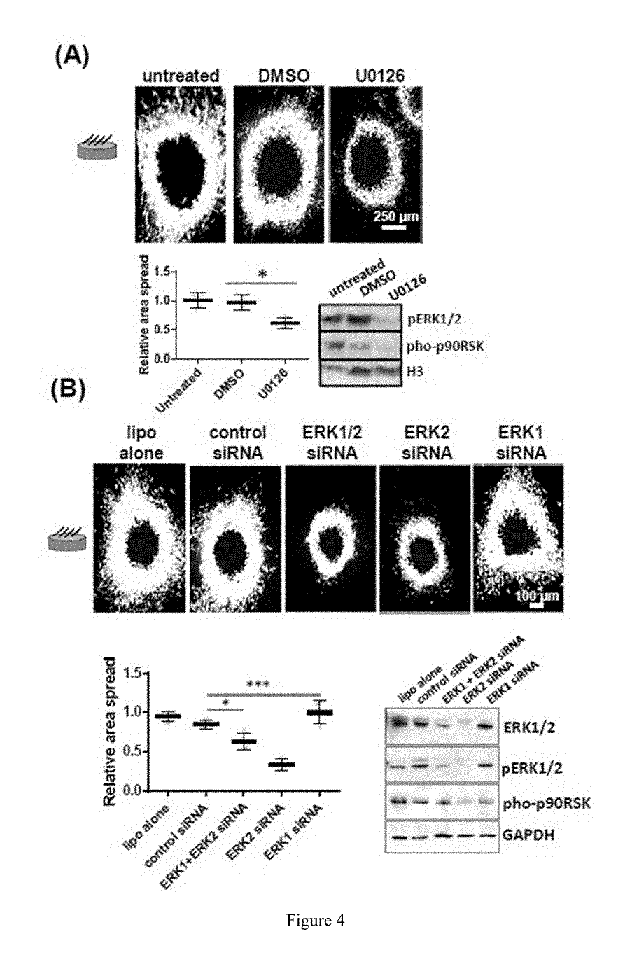

Since mitogen-activated protein kinase, known as MEK, is known to regulate ERK1/2 downstream to KRAS, we next cultured the kRAS cell spheroids within CAF-CDMs that were produced onto patho stiffness gels, in the presence or absence of the MEK inhibitor U0126, and measured area spreads (as described earlier) as well as nuclear levels of pERK1/2 using fractionated cell lysates via Immunoblot. The results indicated that addition of U0126 rendered a significant loss of pERK1/2 at nuclear locations that was concomitant of a significant reduction in KRAS spheroid spread (see, FIG. 4, Panel A). Note that phosphorylation levels of p90 ribosomal S6 kinase (pho-p90RSK), which act downstream to ERK2, were also downregulated in response to MEK inhibition, suggesting the possibility of a role for ERK2 in regulation of patho stiffness generated CAF-CDM induced kRAS cell invasion. Therefore, to distinguish between specific roles for ERK1 vs. ERK2, we transiently knocked down each or both kinases, in KRAS PDAC cells, using specific siRNAs and compared results to equal amounts of scrambled (e.g., non-specific) siRNA. Effective protein downregulations were confirmed via immunoblotting (see, FIG. 4, Panel B) and changes in KRAS spheroid spread, induced by CAF-CDM produced onto patho gels, were measured. The results showed that while ERK1 downregulation provided no appreciable effects, lowering ERK2 levels resulted in significant decreased KRAS spheroid-spread areas, akin to levels observed in response to restrictive CDMs (e.g., CAF-ECMs produced on physio substrate stiffness). These data suggested that ERK2 function, but not ERK1 promotes KRAS spheroid spread. Interestingly, downregulation of ERK2 alone provided a more effective spheroid spread suppression than the co-downregulation of both ERK1 and ERK2. This suggested a possible compensatory role of ERK2 upon ERK1 loss, as indicated by the upregulation of ERK2 in response to downregulation of ERK1; evident in FIG. 4, Panel B and as previously suggested. Again, pho-p90RSK levels, downstream to ERK2, were used to confirm the effective inhibition of the KRAS/ERK2 pathway (see, FIG. 4, Panel B). Together, these results suggested that ERK2, perhaps via pho-p90RSK, is essential for tumorigenic responses induced by CAF-CDMs upon oncogenic KRAS-driven pancreatic cancer cells. ERK2, but not ERK1, downregulation simulates kRAS cell spheroid inhibition akin to restriction levels attained by isotropic CDMs.

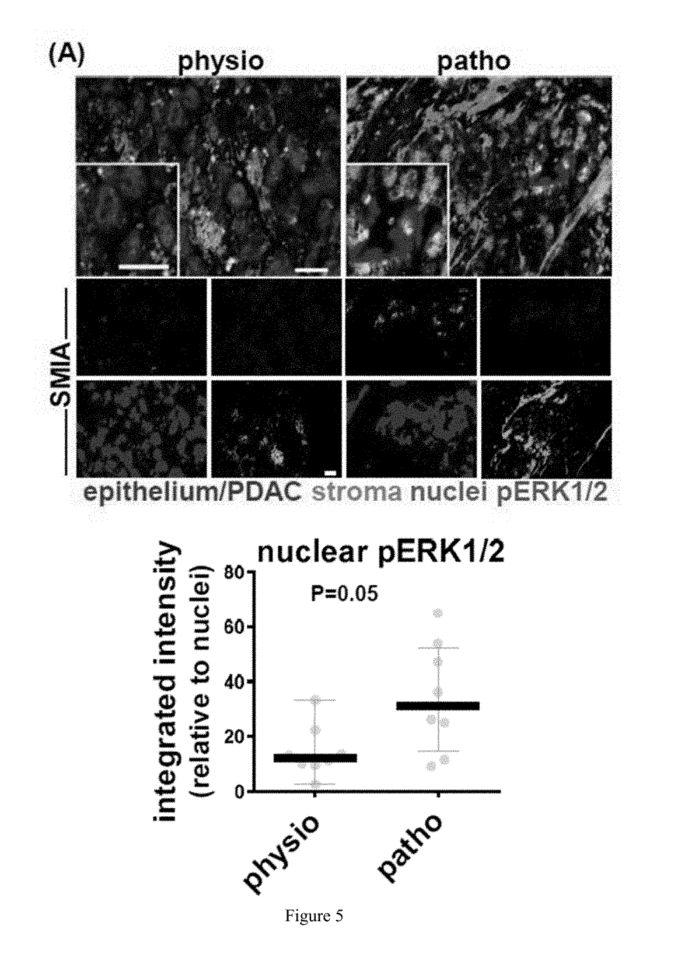

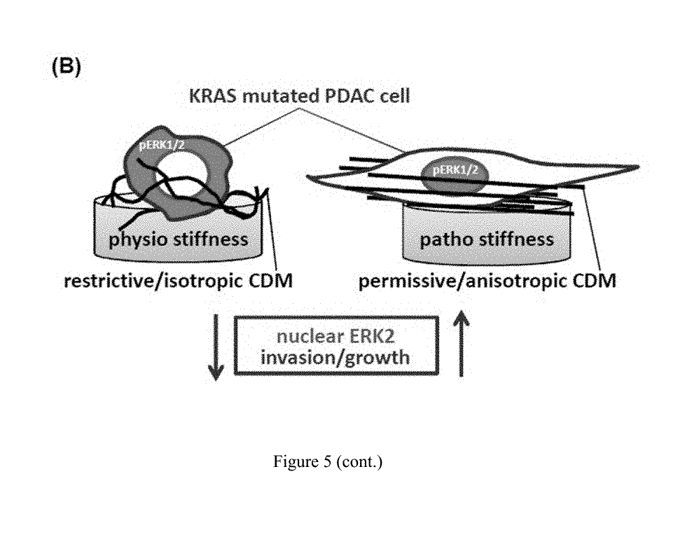

Referring specifically to FIG. 4 (Panels A-B), anisotropic CDM-induced PDAC invasion is regulated by pERK2 is shown. Panel A shows KRAS cells invading through were treated overnight with 20 μM of U0126, to inhibit MEK1/2 upstream to pERK1/2, and phospho-p90RSK, downstream to pERK2. Immunoblots of nuclear fractions are shown. H3 histone served as loading control. Untreated or vehicle treated cells (DMSO) conditions were used as negative controls. Images represent masks generated as in FIG. 5. Output numbers, obtained using SMIA-CUKIE 2.1.0 as above, were plotted and are shown in the corresponding graph. Note that the effect of ERK inhibition is greater on 2D gels (2D patho) compared to 3D CAF-CDM produced onto patho gels. Panel B shows ERK1 and/or ERK2 or scrambled (control) siRNAs were transfected to RFP-expressing KRAS cells and spheroids were allowed to spread onto CAF-CDMs that were generated on patho stiffness gels as before. Lysates were analyzed via immunoblotting (gel top) or images were acquired to assess PDAC area spreads. Representative area spread images are shown while the graph represents a summary of the measured results. Note that ERK2 downregulation, but not ERK1, lead to significant inhibition of CDM induced KRAS cell spread.

To validate our in silico and in vitro findings, we examined PDAC nuclei levels of pERK1/2 vs. levels in matching normal pancreatic epithelial nuclei. For this, we used our recently published multi-color SMI analysis approach. Images shown in FIG. 5, Panel A demonstrated a high occurrence in nuclear pERK1/2 in PDAC and to a lesser extent in normal pancreatic epithelial cells. SMIA-CUKIE 2.1.0 (see, world wide web at “github.com/cukie/SMIA/releases”) was used to perform quantitative analysis of four biomarkers that were simultaneously labeled (red epithelia/tumor, gray stroma, blue nuclei and green pERK1/2), in normal or pathological PDAC samples. Quantification of pERK1/2 levels localized at nuclear epithelial (in normal) vs. nuclear PDAC-tumor pixel areas using 8 cases, suggested a significant increased trend (about 3-fold; p=0.05) of nuclear pERK1/2 localization in PDAC compared to normal, patient matched, samples. These results agreed with our in vitro observations and suggest that in pancreatic cancer tissue, cancer cells display high levels of nuclear pERK1/2. More importantly, our results suggested the possibility that normal/restrictive stroma is capable of maintaining pERK1/2 away from epithelial nuclei.

Referring specifically to FIG. 5 (Panels A-B), normal stroma maintains pERK1/2 away from pancreatic epithelial nuclei. Panel A shows representative examples of pancreatic normal and PDAC samples that were analyzed using SMI followed by SMIA-CUKIE 2.1.0 as published (Kaukonen et al., 2016, Nature communications, 7, 12237). Top panels are merged images of a representative matching normal and PDAC sample showing epithelium/tumoral cells (red), nuclei (blue), stromal cells (grey) and pERK1/2 (green). The monochromatic panels shown below indicate masks generated by the SMIA-CUKIE software (SMIA) and the levels or pERK1/2 located only at epithelial or tumoral nuclei areas (e.g., omitting all cytosolic or stromal positive pixels (green)). Epithelial and/or tumoral nuclei masks are shown in blue, while tumoral and stromal masks are shown in the bottom panels, in red and gray, respectively. Graphs summarizing SMIA-CUKIE-generated data outputs represent the measured intensity levels of nuclear epithelial (normal) and tumoral (PDAC) activated ERK1/2 (pERK1/2). P value is indicated. Panel B provides a summary depiction showing that isotropic CDMs direct pERK1/2 (green) to cytosolic locations restricting PDAC cell behaviors.

Despite reports suggesting that mechanical properties of desmoplastic stromal matrix play central roles in regulation of tumorigenesis, the particular mechanisms that enable stromal ECM production with tumor restrictive capabilities remain mostly elusive. The present study was designed, in part, to determine whether changing substrate stiffness influences the ability of CAFs to biomechanically remodel CAF-derived CDM so that the matrix becomes restrictive to kRAS driven pancreatic cancer. Some major discoveries in this study are as follows: First, via experimental and mathematical modeling approaches we demonstrated that changing the underlying substrate stiffness of CAFs results in alteration of ECM phenotypic properties such as ECM indentation modulus and anisotropy. Second, CAF derived CDM generated on physio gel is tumor-restrictive and the tumor restriction effects are mediated through loss of nuclear ERK-2. Finally, the significance of the 3D CDM presented model was demonstrated in vivo using patient matched samples representing PDAC and normal pancreas. Taken together, our data suggest that substrate stiffness triggers mechanical changes in CAFs-derived ECM, which may ultimately manifest a change in biological function within kRAS pancreatic cancer cells.

As means to manipulate CAF-ECM mechanical characteristics, we chose to alter the underlying rigidity of CAFs. By means of mathematical modeling, we were able to demonstrate that substrate stiffness dictates a biphasic change in CAF (and control fibroblast) shape, which in turn dictates the anisotropy levels of fibroblastic CDM fibers. Our predictions are line with other studies that suggest that that matrix stiffness dictates a biphasic change in cell responses such as motility and proliferation. Interestingly, the measured indentation moduli of all CDMs also did not directly correlate with the stiffness of the underlying substrate and the CDMs are softer, in some cases by levels of magnitude, than their underlying substrates.

We observed that matrix alignment was the main reason for the inducing crash restriction, but not matrix stiffness. An increase in ECM anisotropy in activated fibroblasts is known to facilitate cancer invasion and metastasis.

We observed that rearrangements in localization of nuclear pERK1/2, as opposed to increased levels, of pERK1/2 correlated with CDM induced PDAC cell proliferation and fast directional motility. Therefore, our study suggests that restrictive CDMs prevent the nuclear accumulation of pERK1/2. Interestingly, we observed that ERK2 and not ERK1 is responsible for regulating the observed ECM-induced PDAC cell responses. Our study suggests that nuclear ERK2, via its canonical downstream effector p90RSK, may be implicated in ECM induced oncogenic KRAS-driven PDAC cells responses, such as invasion.

Overall, these studies demonstrate that by changing the underlying substrate stiffness, tumor-restrictive isotropic (as opposed to anisotropic) CAF-CDMs could be produced. Furthermore, physical changes in desmoplastic ECM, especially anisotropic topographical conformations, can act via ERK2 to sustain oncogenic KRAS activity regulated proliferative and invasive characteristics in PDAC cells. Hence, the potential therapeutic reprogramming of stromal ECM and/or targeting tumoral ERK2 may provide new future means to treat pancreatic cancer.

Various modifications of the described subject matter, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety.

What is claimed is:

1. A cell-derived matrix (CDM)/gel system comprising:
a substrate having a gel on a surface thereof; and
a CDM on the surface of the gel.

2. The system according to claim 1, wherein the substrate is a petri dish, a multi-well plate, or a coverslip.

3. The system according to claim 1, wherein the substrate is activated.

4. The system according to claim 3, wherein the substrate is activated with 3-aminopropyl triethoxysilane (APTES) or 3-aminopropyldimethylethoxysilane (APDMES).

5. The system according to claim 1, wherein the gel is a polyacrylamide gel, a polydimethylsiloxane (PDMS) gel, or a polyethylene glycol (PEG) gel.

6. The system according to claim 5, wherein the polyacrylamide gel comprises acrylamide and N,N′-methylenebiacrylamide.

7. The system according to claim 1, wherein the gel is at least 100 μm thick.

8. The system according to claim 1, wherein the Young's moduli of the gel is from about 0.5 kPa to about 10 kPa.

9. The system according to claim 1, wherein the gel is conjugated to a protein.

10. The system according to claim 1, further comprising cell media.

11. The system according to claim 1, wherein the cells producing the CDM are normal cells or cancer associated cells.

12. A method of preparing the CDM/gel system according to claim 1 comprising:
forming the gel on a surface of a substrate; and
forming the CDM on the surface of the gel.

13. The method according to claim 12 wherein the gel is formed on the surface of the substrate by a method comprising:
chemically activating the substrate;
mixing polymers in ratios to provide the gel with a Young's moduli of from about 0.5 kPa to about 10 kPa to form a polymer solution;
initiating polymerization of the polymer solution; and
contacting the activated surface of the substrate with the polymer solution.

14. The method according to claim 12, further comprising placing a second substrate on top of the polymer solution on the activated surface of the substrate.

15. The method according to claim 14, further comprising removing the second substrate.

16. The method according to claim 12, further comprising conjugating the gel with a protein.

17. The method according to claim 12, wherein the CDM is formed on the surface of the gel by a method comprising:
adding cell growth media and cells to the gel; and
incubating the substrate having the gel and cells under conditions sufficient for the development of the ECM.

18. The method according to claim 17, further comprising extracting the CDM from the gel.

19. A method of screening an agent or a combination of agents for anti-cancer activity or anti-fibrotic activity comprising:
contacting the ECM/gel system according to claim 1 with the agent or combination of agents; and
assaying the ECM for at least one anti-cancer characteristic or at least one anti-fibrotic characteristic, whereby if the agent induces at least one anti-cancer characteristic or anti-fibrotic characteristic on the ECM, the agent is a potential anti-cancer or anti-fibrotic drug.

20. The method according to claim 19, wherein the at least one anti-cancer characteristic or anti-fibrotic characteristic of the extracellular matrix is selected from decreased formation of highly anisotropic collagen fibers, decreased stiffness, decrease in cell spheroid migration, decrease in cell proliferation, decreased spindle shaped morphology, decreased levels of alpha-smooth muscle actin, decreased cell-aspect ratio, decreased matric indentation modulus, increased apoptosis, decreased cell survival, and differentiation.
